SEQUENCE LISTING (5 sequences)

SEQ ID NO: 1 - DNA, 13121 bp, Artificial Sequence
  Expression plasmid
ttaagttattggtatgactggttttaagcgcaaaaaaagttgctttttcgtacctattaatgtatcgttttagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaagaccacattaaaaaatgtggtcttttgtgtttttttaaaggatttgagcgtagcgaaaaatccttttctttcttatcttgataataagggtaactattgccgatcgtccattccgacagcatcgccagtcactatggcgtgctgctagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcaggccttaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatcaaaatcgtctccctccgtttgaatatttgattgatcgtaaccagatgaagcactctttccactatccctacagtgttatggcttgaacaatcacgaaacaataattggtacgtacgatctttcagccgactcaaacatcaaatcttacaaatgtagtctttgaaagtattacatatgtaagatttaaatgcaaccgttttttcggaaggaaatgatgacctcgtttccaccggaattagcttggtaccaaaggaggtaaggatcactagaaaattttttaaaaaatctcttgacattggaagggagatatgttattataagaattgcggaattgtgagcggataacaattcccatatagattaactaataaggaggacaaacatgaaagaaatcaaaatccaaaacatcatcatcagcgaagaaaaagcgccgctggttgtgccggaaatcggcattaatcataacggatctttagaactggctaaaatcatggttgatgcagcgttttcagctggagccaaaatcatcaaacatcaaacacatatcgtcgaagatgaaatgagcaaagctgcaaagaaagttatcccgggcaacgctaaaatctctatctacgaaatcatgcagaaatgcgctttagattacaaagatgaacttgccctgaaagaatacacagaaaaactgggacttgtgtatcttagcacaccgttttcaagagcaggcgcgaatcgcttagaagatatgggagtctctgcatttaaaatcggctcaggagaatgtaataactatccgctgatcaaacatattgcagcgtttaaaaaaccgatgattgtgtctacaggcatgaactcaatcgaaagcattaaaccgacagtgaaaatcctgcttgataacgaaatcccgtttgtcctgatgcatacaacaaacctgtatccgacaccgcataatcttgttagattaaacgccatgctggaactgaagaaagaatttagctgcatggtgggactttctgatcatacaacagataatctggcatgccttggcgcggtcgttcttggagcgtgtgtcttagaaagacattttacagattcaatgcatcgcagcggaccggatattgtttgttctatggatacaaaagccttaaaagaactgatcattcaatcagaacagatggcaatcattcgcggcaataacgaatcaaagaaagcagccaaacaagaacaggttacaatcgattttgcttttgcctctgtggtctcaatcaaagatattaagaaaggcgaagttctgtcaatggataatatttgggtgaaaagaccgggacttggcggaatcagcgcagcggaatttgaaaatattctgggcaagaaagcactgcgcgatattgaaaacgatgcgcagttaagctatgaagattttgcttaagctcttaaggaggattttagaatgaagaaaattctgtttatcacaggctcaagagcggattactctaaaatcaaatcacttatgtaccgcgtccaaaattcaagcgaatttgaactttacatctttgctacaggcatgcatctgagcaaaaactttggatacacagttaaagaattatataaaaatggctttaaaaacatctacgaatttatcaactacgataaatattatcagacagataaagccctggcaacaacaattgatggattttcaagatatgcgaacgaattaaaaccggatctgattgttgtgcatggcgatcgcatcgaaccgcttgcagcggctattgtcggagcccttaacaacatcctggttgcacatatcgaaggcggagaaattagcggaacaatcgatgattctttaagacatgcgatttcaaaacttgctcatatccatctggtgaacgatgaatttgcaaaaagacgccttatgcaattaggcgaagatgaaaaatcaatctttatcatcggatctccggatctggaactgcttaacgataacaaaatctcacttagcgaagccaaaaaatactacgatattaactacgaaaactatgcattactgatgtttcatccggtcacaacagaaatcacatctatcaaaaaccaagccgataacctggtgaaagcacttatccagtcaaacaaaaactacatcgtcatctatccgaataacgatctgggctttgaactgatccttcagagctatgaagaatttaaaaacaacccgagatttaaactgtttccgtctctgcgctttgaatactttatcacacttctgaaaaacgccgattttattatcggaaactcttcatgcatcctgaaagaagcgttatacctgaaaacagctggcattctggttggatcaagacaaaatggccgcttaggaaatgaaaacacactgaaagtgaatgcgaacagcgatgaaatccttaaagctatcaacacaatccataaaaaacaggatttattttctgctaaactggaaattcttgatagctctaaactgtttttcgaatatcttcaatcaggcgatttctttaaacttagcacacagaaagtttttaaagatattaaataaaaaggaggaactactatgtcacttgcaattatcccggcgagaggcggaagcaaaggcatcaaaaacaaaaacctggttctgcttaacaacaaaccgcttatctactacacaatcaaagcagcgctgaatgctaaatctatttcaaaagttgtggtctcaagcgatagcgatgaaatccttaactacgccaaatctcaaaacgtggatattctgaaaagaccgatctctcttgcacaggatgatacaacatcagataaagtcttactgcatgcgctgaaattttacaaagattacgaagatgttgtgtttttacaaccgacatctccgctgcgcacaaacattcatatcaacgaagcatttaacctgtacaaaaattcaaacgctaatgccctgattagcgtctctgaatgcgataacaaaatccttaaagcatttgtttgcaacgattgtggcgatttagccggaatttgtaatgatgaatatccgtttatgccgcgccagaaactgccgaaaacatatatgagcaacggagcgatctacatccttaaaatcaaagaatttctgaacaacccgtcatttctgcaaagcaaaacaaaacattttcttatggatgaatcatcatcactggatattgattgcctggaagatttaaagaaagttgaacaaatttggaaaaaataaagattaactaataaggaggacaaacatggataaatttgctgaacatgaaattccgaaagctgttatcgtggccggcaacggagaatctctttcacaaatcgattacagactgcttccgaaaaactatgatgtctttagatgcaaccagttttactttgaagaacgctattttcttggcaacaaaatcaaagcagttttctttacaccgggagtgtttttagaacaatactacacactgtaccatcttaaacgcaacaacgaatactttgtcgataacgttatcctgtcatcatttaaccatccgacagttgatctggaaaaatcacaaaaaatccaggctctgtttattgatgtgatcaacggctacgaaaaatacctgtctaaactgacagcctttgatgtttatctgagatacaaagaactttacgaaaaccaacgcattacatcaggagtgtatatgtgcgccgtcgcaatcgcgatgggctatacagatatttaccttacaggaatcgatttttatcaggcaagcgaagaaaactacgcgtttgataacaaaaaaccgaacatcatcagattactgccggattttcgcaaagaaaaaacattattttcatatcatagcaaagatattgatcttgaagcattatcttttctgcaacagcattaccatgttaacttttacagcatttctccgatgtctccgctgtcaaaacattttccgatcccgacagtggaagatgattgtgaaacaacatttgtcgcgccgctgaaagaaaactacatcaacgatattcttttaccgccgcattttgtctatgaaaaacttggcgttgataaacttgcagcggctttagaacatcatcatcatcatcattaaagtgatagcggtaccattataggtaagagaggaatgtacacatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgcactcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaaggatccatgtctagagtcgacgtccccggggcagcccgcctaatgagcgggcttttttcacgtcccaggcatcaaataaaacgaaaggctcagtcgaaagactgggcctttcgttttatctgttgtttgtcggtgaacgctctctactagagtcacactggctcaccttcgggtgggcctttctgcgtttatacccggggcagcccgcctaatgagcgggcttttttcacgtcacgcgtccatggagatctttgtctgcaactgaaaagtttataccttacctggaacaaatggttgaaacatacgaggctaatatcggcttattaggaatagtccctgtactaataaaatcaggtggatcagttgatcagtatattttggacgaagctcggaaagaatttggagatgacttgcttaattccacaattaaattaagggaaagaataaagcgatttgatgttcaaggaatcacggaagaagatactcatgataaagaagctctaaaactattcaataaccttacaatggaattgatcgaaagggtggaaggttaatggtacgaaaattaggggatctacctagaaagccacaaggcgataggtcaagcttaaagaacccttacatggatcttacagattctgaaagtaaagaaacaacagaggttaaacaaacagaaccaaaaagaaaaaaagcattgttgaaaacaatgaaagttgatgtttcaatccataataagattaaatcgctgcacgaaattctggcagcatccgaagggaattcatattacttagaggatactattgagagagctattgataagatggttgagacattacctgagagccaaaaaactttttatgaatatgaattaaaaaaaagaaccaacaaaggctgagacagactccaaacgagtctgtttttttaaaaaaaatattaggagcattgaatatatattagagaattaagaaagacatgggaataaaaatattttaaatccagtaaaaatatgataagattatttcagaatatgaagaactctgtttgtttttgatgaaaaaacaaacaaaaaaaatccacctaacggaatctcaatttaactaacagcggccaaactgagaagttaaatttgagaaggggaaaaggcggatttatacttgtatttaactatctccattttaacattttattaaaccccatacaagtgaaaatcctcttttacactgttcctttaggtgatcgcggagggacattatgagtgaagtaaacctaaaaggaaatacagatgaattagtgtattatcgacagcaaaccactggaaataaaatcgccaggaagagaatcaaaaaagggaaagaagaagtttattatgttgctgaaacggaagagaagatatggacagaagagcaaataaaaaacttttctttagacaaatttggtacgcatataccttacatagaaggtcattatacaatcttaaataattacttctttgatttttggggctattttttaggtgctgaaggaattgcgctctatgctcacctaactcgttatgcatacggcagcaaagacttttgctttcctagtctacaaacaatcgctaaaaaaatggacaagactcctgttacagttagaggctacttgaaactgcttgaaaggtacggttttatttggaaggtaaacgtccgtaataaaaccaaggataacacagaggaatccccgatttttaagattagacgtaaggttcctttgctttcagaagaacttttaaatggaaaccctaatattgaaattccagatgacgaggaagcacatgtaaagaaggctttaaaaaaggaaaaagagggtcttccaaaggttttgaaaaaagagcacgatgaatttgttaaaaaaatgatggatgagtcagaaacaattaatattccagaggccttacaatatgacacaatgtatgaagatatactcagtaaaggagaaattcgaaaagaaatcaaaaaacaaatacctaatcctacaacatcttttgagagtatatcaatgacaactgaagaggaaaaagtcgacagtactttaaaaagcgaaatgcaaaatcgtgtctctaagccttcttttgatacctggtttaaaaacactaagatcaaaattgaaaataaaaattgtttattacttgtaccgagtgaatttgcatttgaatggattaagaaaagatatttagaaacaattaaaacagtccttgaagaagctggatatgttttcgaaaaaatcgaactaagaaaagtgcaataaactgctgaagtatttcagcagttttttttatttagaaatagtgaaaaaaatataatcagggaggtatcaatatttaatgagtactgatttaaatttatttagactggaattaataattaacacgtagactaattaaaatttaatgagggataaagaggatacaaaaatattaatttcaatccctattaaattttaacaagggggggattaaaatttaattagaggtttatccacaagaaaagaccctaataaaatttttactagggttataacactgattaatttcttaatgggggagggattaaaatttaatgacaaagaaaacaatcttttaagaaaagcttttaaaagataataataaaaagagctttgcgattaagcaaaactctttactttttcattgacattatcaaattcatcgatttcaaattgttgttgtatcataaagttaattctgttttgcacaaccttttcaggaatataaaacacatctgaggcttgttttataaactcagggtcgctaaagtcaatgtaacgtagcatatgatatggtatagcttccacccaagttagcctttctgcttcttctgaatgtttttcatatacttccatgggtatctctaaatgattttcctcatgtagcaaggtatgagcaaaaagtttatggaattgatagttcctctctttttcttcaacttttttatctaaaacaaacactttaacatctgagtcaatgtaagcataagatgtttttccagtcataatttcaatcccaaatcttttagacagaaattctggacgtaaatcttttggtgaaagaatttttttatgtagcaatatatccgatacagcaccttctaaaagcgttggtgaatagggcattttacctatctcctctcattttgtggaataaaaatagtcatattcgtccatctacctatcctattatcgaacagttgaactttttaatcaaggatcagtcctttttttcattattcttaaactgtgctcttaactttaacaactcgatttgtttttccagatctcgagggtaactagcctcgccgatcccgcaagaggcccggcagtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcatgc

SEQ ID NO: 2 - DNA, 11125 bp, Artificial Sequence
  Plasmid
ttaagttattggtatgactggttttaagcgcaaaaaaagttgctttttcgtacctattaatgtatcgttttagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaagaccacattaaaaaatgtggtcttttgtgtttttttaaaggatttgagcgtagcgaaaaatccttttctttcttatcttgataataagggtaactattgccgatcgtccattccgacagcatcgccagtcactatggcgtgctgctagcattttacatttttagaaatgggcgtgaaaaaaagcgcgcgattatgtaaaatataaagattaactaataaggaggacaaacatgaaagaaatcaaaatccaaaacatcatcatcagcgaagaaaaagcgccgctggttgtgccggaaatcggcattaatcataacggatctttagaactggctaaaatcatggttgatgcagcgttttcagctggagccaaaatcatcaaacatcaaacacatatcgtcgaagatgaaatgagcaaagctgcaaagaaagttatcccgggcaacgctaaaatctctatctacgaaatcatgcagaaatgcgctttagattacaaagatgaacttgccctgaaagaatacacagaaaaactgggacttgtgtatcttagcacaccgttttcaagagcaggcgcgaatcgcttagaagatatgggagtctctgcatttaaaatcggctcaggagaatgtaataactatccgctgatcaaacatattgcagcgtttaaaaaaccgatgattgtgtctacaggcatgaactcaatcgaaagcattaaaccgacagtgaaaatcctgcttgataacgaaatcccgtttgtcctgatgcatacaacaaacctgtatccgacaccgcataatcttgttagattaaacgccatgctggaactgaagaaagaatttagctgcatggtgggactttctgatcatacaacagataatctggcatgccttggcgcggtcgttcttggagcgtgtgtcttagaaagacattttacagattcaatgcatcgcagcggaccggatattgtttgttctatggatacaaaagccttaaaagaactgatcattcaatcagaacagatggcaatcattcgcggcaataacgaatcaaagaaagcagccaaacaagaacaggttacaatcgattttgcttttgcctctgtggtctcaatcaaagatattaagaaaggcgaagttctgtcaatggataatatttgggtgaaaagaccgggacttggcggaatcagcgcagcggaatttgaaaatattctgggcaagaaagcactgcgcgatattgaaaacgatgcgcagttaagctatgaagattttgcttaagctcttaaggaggattttagaatgaagaaaattctgtttatcacaggctcaagagcggattactctaaaatcaaatcacttatgtaccgcgtccaaaattcaagcgaatttgaactttacatctttgctacaggcatgcatctgagcaaaaactttggatacacagttaaagaattatataaaaatggctttaaaaacatctacgaatttatcaactacgataaatattatcagacagataaagccctggcaacaacaattgatggattttcaagatatgcgaacgaattaaaaccggatctgattgttgtgcatggcgatcgcatcgaaccgcttgcagcggctattgtcggagcccttaacaacatcctggttgcacatatcgaaggcggagaaattagcggaacaatcgatgattctttaagacatgcgatttcaaaacttgctcatatccatctggtgaacgatgaatttgcaaaaagacgccttatgcaattaggcgaagatgaaaaatcaatctttatcatcggatctccggatctggaactgcttaacgataacaaaatctcacttagcgaagccaaaaaatactacgatattaactacgaaaactatgcattactgatgtttcatccggtcacaacagaaatcacatctatcaaaaaccaagccgataacctggtgaaagcacttatccagtcaaacaaaaactacatcgtcatctatccgaataacgatctgggctttgaactgatccttcagagctatgaagaatttaaaaacaacccgagatttaaactgtttccgtctctgcgctttgaatactttatcacacttctgaaaaacgccgattttattatcggaaactcttcatgcatcctgaaagaagcgttatacctgaaaacagctggcattctggttggatcaagacaaaatggccgcttaggaaatgaaaacacactgaaagtgaatgcgaacagcgatgaaatccttaaagctatcaacacaatccataaaaaacaggatttattttctgctaaactggaaattcttgatagctctaaactgtttttcgaatatcttcaatcaggcgatttctttaaacttagcacacagaaagtttttaaagatattaaataaaaaggaggaactactatgtcacttgcaattatcccggcgagaggcggaagcaaaggcatcaaaaacaaaaacctggttctgcttaacaacaaaccgcttatctactacacaatcaaagcagcgctgaatgctaaatctatttcaaaagttgtggtctcaagcgatagcgatgaaatccttaactacgccaaatctcaaaacgtggatattctgaaaagaccgatctctcttgcacaggatgatacaacatcagataaagtcttactgcatgcgctgaaattttacaaagattacgaagatgttgtgtttttacaaccgacatctccgctgcgcacaaacattcatatcaacgaagcatttaacctgtacaaaaattcaaacgctaatgccctgattagcgtctctgaatgcgataacaaaatccttaaagcatttgtttgcaacgattgtggcgatttagccggaatttgtaatgatgaatatccgtttatgccgcgccagaaactgccgaaaacatatatgagcaacggagcgatctacatccttaaaatcaaagaatttctgaacaacccgtcatttctgcaaagcaaaacaaaacattttcttatggatgaatcatcatcactggatattgattgcctggaagatttaaagaaagttgaacaaatttggaaaaaataaagtcaatgtatgaatggatacgggatatgaatcaataagtacgtgaaagagaaaagcaacccagatatgatagggaacttttctctttcttgttttacattgaatctttacaatcctattgatataatctaagctagtgtattttgcgtttaatagtggagaaaagctagcgattaactaataaggaggacaaacatggataaatttgctgaacatgaaattccgaaagctgttatcgtggccggcaacggagaatctctttcacaaatcgattacagactgcttccgaaaaactatgatgtctttagatgcaaccagttttactttgaagaacgctattttcttggcaacaaaatcaaagcagttttctttacaccgggagtgtttttagaacaatactacacactgtaccatcttaaacgcaacaacgaatactttgtcgataacgttatcctgtcatcatttaaccatccgacagttgatctggaaaaatcacaaaaaatccaggctctgtttattgatgtgatcaacggctacgaaaaatacctgtctaaactgacagcctttgatgtttatctgagatacaaagaactttacgaaaaccaacgcattacatcaggagtgtatatgtgcgccgtcgcaatcgcgatgggctatacagatatttaccttacaggaatcgatttttatcaggcaagcgaagaaaactacgcgtttgataacaaaaaaccgaacatcatcagattactgccggattttcgcaaagaaaaaacattattttcatatcatagcaaagatattgatcttgaagcattatcttttctgcaacagcattaccatgttaacttttacagcatttctccgatgtctccgctgtcaaaacattttccgatcccgacagtggaagatgattgtgaaacaacatttgtcgcgccgctgaaagaaaactacatcaacgatattcttttaccgccgcattttgtctatgaaaaacttggcgttgataaacttgcagcggctttagaacatcatcatcatcatcattaaagtgatagcggtaccattataggtaagagaggaatgtacacatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgcactcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaaggatccatgtctagagtcgacgtccccggggcagcccgcctaatgagcgggcttttttcacgtcccaggcatcaaataaaacgaaaggctcagtcgaaagactgggcctttcgttttatctgttgtttgtcggtgaacgctctctactagagtcacactggctcaccttcgggtgggcctttctgcgtttatagaattcatattacttagaggatactattgagagagctattgataagatggttgagacattacctgagagccaaaaaactttttatgaatatgaattaaaaaaaagaaccaacaaaggctgagacagactccaaacgagtctgtttttttaaaaaaaatattaggagcattgaatatatattagagaattaagaaagacatgggaataaaaatattttaaatccagtaaaaatatgataagattatttcagaatatgaagaactctgtttgtttttgatgaaaaaacaaacaaaaaaaatccacctaacggaatctcaatttaactaacagcggccaaactgagaagttaaatttgagaaggggaaaaggcggatttatacttgtatttaactatctccattttaacattttattaaaccccatacaagtgaaaatcctcttttacactgttcctttaggtgatcgcggagggacattatgagtgaagtaaacctaaaaggaaatacagatgaattagtgtattatcgacagcaaaccactggaaataaaatcgccaggaagagaatcaaaaaagggaaagaagaagtttattatgttgctgaaacggaagagaagatatggacagaagagcaaataaaaaacttttctttagacaaatttggtacgcatataccttacatagaaggtcattatacaatcttaaataattacttctttgatttttggggctattttttaggtgctgaaggaattgcgctctatgctcacctaactcgttatgcatacggcagcaaagacttttgctttcctagtctacaaacaatcgctaaaaaaatggacaagactcctgttacagttagaggctacttgaaactgcttgaaaggtacggttttatttggaaggtaaacgtccgtaataaaaccaaggataacacagaggaatccccgatttttaagattagacgtaaggttcctttgctttcagaagaacttttaaatggaaaccctaatattgaaattccagatgacgaggaagcacatgtaaagaaggctttaaaaaaggaaaaagagggtcttccaaaggttttgaaaaaagagcacgatgaatttgttaaaaaaatgatggatgagtcagaaacaattaatattccagaggccttacaatatgacacaatgtatgaagatatactcagtaaaggagaaattcgaaaagaaatcaaaaaacaaatacctaatcctacaacatcttttgagagtatatcaatgacaactgaagaggaaaaagtcgacagtactttaaaaagcgaaatgcaaaatcgtgtctctaagccttcttttgatacctggtttaaaaacactaagatcaaaattgaaaataaaaattgtttattacttgtaccgagtgaatttgcatttgaatggattaagaaaagatatttagaaacaattaaaacagtccttgaagaagctggatatgttttcgaaaaaatcgaactaagaaaagtgcaataaactgctgaagtatttcagcagttttttttatttagaaatagtgaaaaaaatataatcagggaggtatcaatatttaatgagtactgatttaaatttatttagactggaattaataattaacacgtagactaattaaaatttaatgagggataaagaggatacaaaaatattaatttcaatccctattaaattttaacaagggggggattaaaatttaattagaggtttatccacaagaaaagaccctaataaaatttttactagggttataacactgattaatttcttaatgggggagggattaaaatttaatgacaaagaaaacaatcttttaagaaaagcttttaaaagataataataaaaagagctttgcgattaagcaaaactctttactttttcattgacattatcaaattcatcgatttcaaattgttgttgtatcataaagttaattctgttttgcacaaccttttcaggaatataaaacacatctgaggcttgttttataaactcagggtcgctaaagtcaatgtaacgtagcatatgatatggtatagcttccacccaagttagcctttctgcttcttctgaatgtttttcatatacttccatgggtatctctaaatgattttcctcatgtagcaaggtatgagcaaaaagtttatggaattgatagttcctctctttttcttcaacttttttatctaaaacaaacactttaacatctgagtcaatgtaagcataagatgtttttccagtcataatttcaatcccaaatcttttagacagaaattctggacgtaaatcttttggtgaaagaatttttttatgtagcaatatatccgatacagcaccttctaaaagcgttggtgaatagggcattttacctatctcctctcattttgtggaataaaaatagtcatattcgtccatctacctatcctattatcgaacagttgaactttttaatcaaggatcagtcctttttttcattattcttaaactgtgctcttaactttaacaactcgatttgtttttccagatctcgagggtaactagcctcgccgatcccgcaagaggcccggcagtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcatgc

SEQ ID NO: 3 - DNA, 13805 bp, Artificial Sequence
  Expression plasmid
ttaagttattggtatgactggttttaagcgcaaaaaaagttgctttttcgtacctattaatgtatcgttttagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaagaccacattaaaaaatgtggtcttttgtgtttttttaaaggatttgagcgtagcgaaaaatccttttctttcttatcttgataataagggtaactattgccgatcgtccattccgacagcatcgccagtcactatggcgtgctgctagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcaggccttaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatcaaaatcgtctccctccgtttgaatatttgattgatcgtaaccagatgaagcactctttccactatccctacagtgttatggcttgaacaatcacgaaacaataattggtacgtacgatctttcagccgactcaaacatcaaatcttacaaatgtagtctttgaaagtattacatatgtaagatttaaatgcaaccgttttttcggaaggaaatgatgacctcgtttccaccggaattagcttggtaccaaaggaggtaaggatcactagaaaattttttaaaaaatctcttgacattggaagggagatatgttattataagaattgcggaattgtgagcggataacaattcccatatagattaactaataaggaggacaaacatgaaagaaatcaaaatccaaaacatcatcatcagcgaagaaaaagcgccgctggttgtgccggaaatcggcattaatcataacggatctttagaactggctaaaatcatggttgatgcagcgttttcagctggagccaaaatcatcaaacatcaaacacatatcgtcgaagatgaaatgagcaaagctgcaaagaaagttatcccgggcaacgctaaaatctctatctacgaaatcatgcagaaatgcgctttagattacaaagatgaacttgccctgaaagaatacacagaaaaactgggacttgtgtatcttagcacaccgttttcaagagcaggcgcgaatcgcttagaagatatgggagtctctgcatttaaaatcggctcaggagaatgtaataactatccgctgatcaaacatattgcagcgtttaaaaaaccgatgattgtgtctacaggcatgaactcaatcgaaagcattaaaccgacagtgaaaatcctgcttgataacgaaatcccgtttgtcctgatgcatacaacaaacctgtatccgacaccgcataatcttgttagattaaacgccatgctggaactgaagaaagaatttagctgcatggtgggactttctgatcatacaacagataatctggcatgccttggcgcggtcgttcttggagcgtgtgtcttagaaagacattttacagattcaatgcatcgcagcggaccggatattgtttgttctatggatacaaaagccttaaaagaactgatcattcaatcagaacagatggcaatcattcgcggcaataacgaatcaaagaaagcagccaaacaagaacaggttacaatcgattttgcttttgcctctgtggtctcaatcaaagatattaagaaaggcgaagttctgtcaatggataatatttgggtgaaaagaccgggacttggcggaatcagcgcagcggaatttgaaaatattctgggcaagaaagcactgcgcgatattgaaaacgatgcgcagttaagctatgaagattttgcttaagctcttaaggaggattttagaatgaagaaaattctgtttatcacaggctcaagagcggattactctaaaatcaaatcacttatgtaccgcgtccaaaattcaagcgaatttgaactttacatctttgctacaggcatgcatctgagcaaaaactttggatacacagttaaagaattatataaaaatggctttaaaaacatctacgaatttatcaactacgataaatattatcagacagataaagccctggcaacaacaattgatggattttcaagatatgcgaacgaattaaaaccggatctgattgttgtgcatggcgatcgcatcgaaccgcttgcagcggctattgtcggagcccttaacaacatcctggttgcacatatcgaaggcggagaaattagcggaacaatcgatgattctttaagacatgcgatttcaaaacttgctcatatccatctggtgaacgatgaatttgcaaaaagacgccttatgcaattaggcgaagatgaaaaatcaatctttatcatcggatctccggatctggaactgcttaacgataacaaaatctcacttagcgaagccaaaaaatactacgatattaactacgaaaactatgcattactgatgtttcatccggtcacaacagaaatcacatctatcaaaaaccaagccgataacctggtgaaagcacttatccagtcaaacaaaaactacatcgtcatctatccgaataacgatctgggctttgaactgatccttcagagctatgaagaatttaaaaacaacccgagatttaaactgtttccgtctctgcgctttgaatactttatcacacttctgaaaaacgccgattttattatcggaaactcttcatgcatcctgaaagaagcgttatacctgaaaacagctggcattctggttggatcaagacaaaatggccgcttaggaaatgaaaacacactgaaagtgaatgcgaacagcgatgaaatccttaaagctatcaacacaatccataaaaaacaggatttattttctgctaaactggaaattcttgatagctctaaactgtttttcgaatatcttcaatcaggcgatttctttaaacttagcacacagaaagtttttaaagatattaaataaaaaggaggaactactatgtcacttgcaattatcccggcgagaggcggaagcaaaggcatcaaaaacaaaaacctggttctgcttaacaacaaaccgcttatctactacacaatcaaagcagcgctgaatgctaaatctatttcaaaagttgtggtctcaagcgatagcgatgaaatccttaactacgccaaatctcaaaacgtggatattctgaaaagaccgatctctcttgcacaggatgatacaacatcagataaagtcttactgcatgcgctgaaattttacaaagattacgaagatgttgtgtttttacaaccgacatctccgctgcgcacaaacattcatatcaacgaagcatttaacctgtacaaaaattcaaacgctaatgccctgattagcgtctctgaatgcgataacaaaatccttaaagcatttgtttgcaacgattgtggcgatttagccggaatttgtaatgatgaatatccgtttatgccgcgccagaaactgccgaaaacatatatgagcaacggagcgatctacatccttaaaatcaaagaatttctgaacaacccgtcatttctgcaaagcaaaacaaaacattttcttatggatgaatcatcatcactggatattgattgcctggaagatttaaagaaagttgaacaaatttggaaaaaataaagattaactaataaggaggacaaacatgaaaagaatcttttgccttgtgtctgcaatcctgctttcagcgtgtaatgataaccagaatacagtcgatgttgtggtctcaacagtgaacgataacgtcatcgaaaacaacacataccaggtcaaaccgatcgatacaccgacaacatttgatagctattcttggattcaaacatgcggcacaccgatccttaaagatgatgaaaaatactcactgtcatttgattttgttgctccggaattagatcaggatgaaaaattttgttttgaatttacaggcgatgttgatggaaaacgctatgtgacacaaacaaatctgacagttgtggcaccgacacttgaagtgtatgtcgatcatgcgagcctgccgtctcttcaacagctgatgaaaatcatccaacagaaaaacgaatactctcagaacgaaagatttatctcatggggccgcatcggacttacagaagataacgctgaaaaactgaatgcccatatttatccgctggctggaaataacacatctcaagaattagtcgatgcagttatcgattacgcggattcaaaaaacagactgaatctggaacttaacacaaacacagctcatagctttccgaacttagccccgattctgcgcattatctcaagcaaaagcaacatcctgatctctaacatcaacctttacgatgatggctcagccgaatacgtgaacctgtacaactggaaagatacagaagataaaagcgtcaaactgtctgattcatttttagttctgaaagattactttaatggaatttcttcagaaaaaccgagcggcatctatggaagatataactggcatcagctgtacaacacatcttactattttctgagaaaagattatctgacagtcgaaccgcaactgcatgatcttcgcgaatatcttggcggatcattaaaacagatgagctgggatggcttttcacaactgagcaaaggagataaagaactgtttctgaacattgttggctttgatcaagaaaaactgcaacaggaatatcaacagagcgaacttccgaattttgtgtttacaggaacaacaacatgggcaggcggagaaacaaaagaatattatgcgcaacagcaagttaacgtcgttaacaacgcaatcaacgaaacatctccgtactacctgggcagagaacatgatcttttctttaaaggacatccgcgcggcggaattatcaatgatattatcctgggctcatttaataacatgattgatattccggcgaaagttagctttgaagtgcttatgatgacaggaatgttaccggatacagtgggcggaattgcatcatcactgtattttagcatcccggccgaaaaagtctcttttatcgtttttacatcaagcgatacaatcacagatcgcgaagatgctctgaaatctccgcttgttcaggtgatgatgacactgggcattgtgaaagaaaaagatgtcctgttttggtcagatttaccggattgctcttcaggagtttgtattgcccaatattaaagtgatagcggtaccattataggtaagagaggaatgtacacatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgcactcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaaggatccatgtctagagtcgacgtccccggggcagcccgcctaatgagcgggcttttttcacgtcccaggcatcaaataaaacgaaaggctcagtcgaaagactgggcctttcgttttatctgttgtttgtcggtgaacgctctctactagagtcacactggctcaccttcgggtgggcctttctgcgtttatacccggggcagcccgcctaatgagcgggcttttttcacgtcacgcgtccatggagatctttgtctgcaactgaaaagtttataccttacctggaacaaatggttgaaacatacgaggctaatatcggcttattaggaatagtccctgtactaataaaatcaggtggatcagttgatcagtatattttggacgaagctcggaaagaatttggagatgacttgcttaattccacaattaaattaagggaaagaataaagcgatttgatgttcaaggaatcacggaagaagatactcatgataaagaagctctaaaactattcaataaccttacaatggaattgatcgaaagggtggaaggttaatggtacgaaaattaggggatctacctagaaagccacaaggcgataggtcaagcttaaagaacccttacatggatcttacagattctgaaagtaaagaaacaacagaggttaaacaaacagaaccaaaaagaaaaaaagcattgttgaaaacaatgaaagttgatgtttcaatccataataagattaaatcgctgcacgaaattctggcagcatccgaagggaattcatattacttagaggatactattgagagagctattgataagatggttgagacattacctgagagccaaaaaactttttatgaatatgaattaaaaaaaagaaccaacaaaggctgagacagactccaaacgagtctgtttttttaaaaaaaatattaggagcattgaatatatattagagaattaagaaagacatgggaataaaaatattttaaatccagtaaaaatatgataagattatttcagaatatgaagaactctgtttgtttttgatgaaaaaacaaacaaaaaaaatccacctaacggaatctcaatttaactaacagcggccaaactgagaagttaaatttgagaaggggaaaaggcggatttatacttgtatttaactatctccattttaacattttattaaaccccatacaagtgaaaatcctcttttacactgttcctttaggtgatcgcggagggacattatgagtgaagtaaacctaaaaggaaatacagatgaattagtgtattatcgacagcaaaccactggaaataaaatcgccaggaagagaatcaaaaaagggaaagaagaagtttattatgttgctgaaacggaagagaagatatggacagaagagcaaataaaaaacttttctttagacaaatttggtacgcatataccttacatagaaggtcattatacaatcttaaataattacttctttgatttttggggctattttttaggtgctgaaggaattgcgctctatgctcacctaactcgttatgcatacggcagcaaagacttttgctttcctagtctacaaacaatcgctaaaaaaatggacaagactcctgttacagttagaggctacttgaaactgcttgaaaggtacggttttatttggaaggtaaacgtccgtaataaaaccaaggataacacagaggaatccccgatttttaagattagacgtaaggttcctttgctttcagaagaacttttaaatggaaaccctaatattgaaattccagatgacgaggaagcacatgtaaagaaggctttaaaaaaggaaaaagagggtcttccaaaggttttgaaaaaagagcacgatgaatttgttaaaaaaatgatggatgagtcagaaacaattaatattccagaggccttacaatatgacacaatgtatgaagatatactcagtaaaggagaaattcgaaaagaaatcaaaaaacaaatacctaatcctacaacatcttttgagagtatatcaatgacaactgaagaggaaaaagtcgacagtactttaaaaagcgaaatgcaaaatcgtgtctctaagccttcttttgatacctggtttaaaaacactaagatcaaaattgaaaataaaaattgtttattacttgtaccgagtgaatttgcatttgaatggattaagaaaagatatttagaaacaattaaaacagtccttgaagaagctggatatgttttcgaaaaaatcgaactaagaaaagtgcaataaactgctgaagtatttcagcagttttttttatttagaaatagtgaaaaaaatataatcagggaggtatcaatatttaatgagtactgatttaaatttatttagactggaattaataattaacacgtagactaattaaaatttaatgagggataaagaggatacaaaaatattaatttcaatccctattaaattttaacaagggggggattaaaatttaattagaggtttatccacaagaaaagaccctaataaaatttttactagggttataacactgattaatttcttaatgggggagggattaaaatttaatgacaaagaaaacaatcttttaagaaaagcttttaaaagataataataaaaagagctttgcgattaagcaaaactctttactttttcattgacattatcaaattcatcgatttcaaattgttgttgtatcataaagttaattctgttttgcacaaccttttcaggaatataaaacacatctgaggcttgttttataaactcagggtcgctaaagtcaatgtaacgtagcatatgatatggtatagcttccacccaagttagcctttctgcttcttctgaatgtttttcatatacttccatgggtatctctaaatgattttcctcatgtagcaaggtatgagcaaaaagtttatggaattgatagttcctctctttttcttcaacttttttatctaaaacaaacactttaacatctgagtcaatgtaagcataagatgtttttccagtcataatttcaatcccaaatcttttagacagaaattctggacgtaaatcttttggtgaaagaatttttttatgtagcaatatatccgatacagcaccttctaaaagcgttggtgaatagggcattttacctatctcctctcattttgtggaataaaaatagtcatattcgtccatctacctatcctattatcgaacagttgaactttttaatcaaggatcagtcctttttttcattattcttaaactgtgctcttaactttaacaactcgatttgtttttccagatctcgagggtaactagcctcgccgatcccgcaagaggcccggcagtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcatgc

SEQ ID NO: 4 - DNA, 10838 bp, Artificial Sequence
  Suicide plasmid
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccagcgggcaaggaaagccttcaatatgtgcagtggtatgtcaactctatgaagatcagcctgtttacaatggcagggtctttgctctgtgtgacgtttacggcctatgcgttttcgcgctttcggtttaaagggaggaaatacgctttaacgctctttttattgctgcagatgattcctcagttttcagctttaattgccttgtttgtgctggcgcaaatcttgggaatgatcaatagccactggctgctaatcttgctttatatcggcggcctgatcccgatgaatacgtatttgatgaaagggtacatggattccattccgatggatttagacgaaagcgccaagattgacggagccagcagcaccagaatcttcttccagatcattctgccattatcaaaaccgatggcggcagtcgtggccatgaacggctttaccggtccgctcggagattttgtgctgtcctcaaccatattgagaacgcctgaatcatatacattgcccgtcggtctattcaatttagtgaatgatgtcatgggggccagctatacgacatttgcggccggagccctgcttatcagcataccggttgccgtcatctttattatgctgcaaaagaattttgtgtccggattaaccgcaggcggaacgaagggctaagagaacaaggaggagaatgtgatgtcaaagcttgaaaaaacgcacgtaacaaaagcaaaatttatgctccatgggggagactacaaccccgatcagtggctggatcggcccgatattttagctgacgatatcaaactgatgaagctttctcatacgaatacgttttctgtcggcaattttacatttttagaaatgggcgtgaaaaaaagcgcgcgattatgtaaaatataaagattaactaataaggaggacaaacatgacagaacagtacatcaaaaacgttgaagtgtatcttgattatgcaacaatcccgacactgaactacttttaccattttacagaaaacaaagatgatattgcgacaattagactgtttggccttggacgctttaacatctctaaatcaatcatcgaatcatatccggaaggcattatcagatattgcccgattatctttgaagatcagacagcttttcaacagctgtttatcacactgcttacagaagattctttttgtcaataccgctttaactttcatattaacctgtttcattcatggaaaatgcttatcccgctgctgcatattatctggcaatttaaacataaagtcctggatattaaacttaacttttatgatgatggctctgaaggactggttacactgtcaaaaatcgaacaaaactattcaagcgaaattcttcagaaaatcatcgatattgatagccaatctttttacgctgataaacttagctttttagatgaagatattgccagatatttatggaatagcctgtttgaatcacattactatcttttaaacgattttctgcttaaaaacgaaaaactgagcttactgaaaaactctatcaaatactgccatatcatggatcttgaacgctatttacaatttacacaggaagaaaaagatttctttaacgaacttctgggcattaacatccagagccttgaagataaaatcaaaatctttcaacaaaagaaaacatttatttttacaggaacaacaatcttttctctgccgaaagaagaagaagaaacactttacagactgcatctgaacgctatccttaactacatccatccgaacggcaaatactttatcggcgatggatttacactggtgatcaaaggacatccgcatcaaaaagaaatgaatagcagactggaaaaatcatttgaaaaagccgtcatgctgccggataacatcccgtttgaaattctttacctgatcggctgcaaaccggataaaattggcggatttgtttcaacaagctacttttcatgtgataagaaaaatattgcggatctgctgtttatcagcgcgagacaggaagaagtgcgcaaaaacgattatctttttaacatccaataccagctgagagatatgatgatcaaaacaggatttatccaagaagaaaaaacacatttttacagcgatattccgatctttatttcttaaaaaggaggaactactatgtcacttgcaattatcccggcgagaggcggaagcaaaggcatcaaaaacaaaaacctggttctgcttaacaacaaaccgcttatctactacacaatcaaagcagcgctgaatgctaaatctatttcaaaagttgtggtctcaagcgatagcgatgaaatccttaactacgccaaatctcaaaacgtggatattctgaaaagaccgatctctcttgcacaggatgatacaacatcagataaagtcttactgcatgcgctgaaattttacaaagattacgaagatgttgtgtttttacaaccgacatctccgctgcgcacaaacattcatatcaacgaagcatttaacctgtacaaaaattcaaacgctaatgccctgattagcgtctctgaatgcgataacaaaatccttaaagcatttgtttgcaacgattgtggcgatttagccggaatttgtaatgatgaatatccgtttatgccgcgccagaaactgccgaaaacatatatgagcaacggagcgatctacatccttaaaatcaaagaatttctgaacaacccgtcatttctgcaaagcaaaacaaaacattttcttatggatgaatcatcatcactggatattgattgcctggaagatttaaagaaagttgaacaaatttggaaaaaataagctcttaaggaggattttagaatgagcacaacaacacaaaatatcccgtggtatcgccatcttaacagagcccagtggcgcgcattttctgcagcgtggttaggctatctgcttgatggatttgattttgttctgattgcactggttcttacagaagtgcaaggcgaatttggacttacaacagtgcaggctgcctctttaatttcagcagcgtttatcagcagatggtttggcggattaatgctgggcgcaatgggagatcgctatggaagacgcctggcgatggtcacatctattgttctgttttcagcgggcacattagcttgcggctttgccccgggatatattacaatgtttatcgcgagacttgtgattggcatgggaatggcaggcgaatatggatcaagcgcgacgtatgttatcgaatcttggccgaaacatctgcgcaataaagcatcaggatttcttattagcggcttttctgtgggagcggttgtggctgcccaagtctattctcttgtcgttccggtttggggatggagagcactgtttttcattggcatccttccgattatctttgctctttggttacgcaaaaatatcccggaagccgaagattggaaagaaaaacatgccggaaaagcacctgtgagaacaatggtcgatattctgtatagaggcgaacatcgcattgctaacatcgttatgacattagcagcggctacagctctgtggttttgctttgccggaaatttacaaaacgccgcaatcgttgctgtgctgggcttactgtgtgcggctatttttatcagctttatggttcagtctgccggaaaacgctggccgacaggcgtgatgttaatggtggtcgttctgtttgcttttctttattcatggccgattcaagcccttttaccgacatacctgaaaacagatttagcttacaatccgcatacagtggccaacgtcctgtttttctcaggctttggagccgcagttggctgctgtgtgggcggatttcttggcgattggttaggaacaagaaaagcgtatgtttgttcactgcttgcgagccagttactgattatcccggtttttgcaatcggcggagcgaatgtctgggttcttggacttttactgtttttccaacagatgttaggccaaggaattgctggcatcttaccgaaactgattggcggatattttgatacagatcagagagcggctggccttggatttacatataatgttggagcactgggcggagcccttgcaccgattatcggagcgttaattgctcaacgcctggatcttggcacagcgcttgcttctttatcatttagcctgacatttgtggtcattcttttaatcggccttgatatgccgtcaagagttcagcgctggttaagaccggaagcactgagaacacatgatgcgattgatggcaaaccgttttcaggagctgtgccgtttggcagcgccaaaaacgatctggtcaaaacaaaatcataaccaggcatcaaataaaacgaaaggctcagtcgaaagactgggcctttcgttttatctgttgtttgtcggtgaacgctctctactagagtcacactggctcaccttcgggtgggcctttctgcgtttatatacgcgttaacccgggcccgcggatggatatgatcagatcctttaactctggcaaccctcaaaattgaatgagacatgctacacctccggataataaatatatataaacgtatatagatttcataaagtctaacacactagacttatttacttcgtaattaagtcgttaaaccgtgtgctctacgaccaaaactataaaacctttaagaactttctttttttacaagaaaaaagaaattagataaatctctcatatcttttattcaataatcgcatccgattgcagtataaatttaacgatcactcatcatgttcatatttatcagagctcgtgctataattatactaattttataaggaggaaaaaatatgggcatttttagtatttttgtaatcagcacagttcattatcaaccaaacaaaaaataagtggttataatgaatcgttaataagcaaaattcatataaccaaattaaagagggttataatgaacgagaaaaatataaaacacagtcaaaactttattacttcaaaacataatatagataaaataatgacaaatataagattaaatgaacatgataatatctttgaaatcggctcaggaaaaggccattttacccttgaattagtaaagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatatggtaatataccttataacataagtacggatataatacgcaaaattgtttttgatagtatagctaatgagatttatttaatcgtggaatacgggtttgctaaaagattattaaatacaaaacgctcattggcattacttttaatggcagaagttgatatttctatattaagtatggttccaagagaatattttcatcctaaacctaaagtgaatagctcacttatcagattaagtagaaaaaaatcaagaatatcacacaaagataaacaaaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagcgaggcaggatcaggacaatgattatatttttgtcatgaatttcacggaagaaaaacagctggtcacgtttgatcagagtgtgaaggacataatgacaggagacatattgtcaggcgacctgacgatggaaaagtatgaagtgagaattgtcgtaaacacacattaggctgatgctccgctcgatatgggcggattcttttttctatagaatgaaaacgcttgctaagtcttggggggatgaaatcatgaaaagcaaagtgaaaatgttctttgcggctgccatcgtgtggagtgcatgtagttcaacaggatatgccgctgccattgagaaggagaagcacgtgtcagagcttcgggcagaggatctttttgttaaaaaagtagaggggatgaacaaggattttatcaaaggggcagatgtatccagcgttattgctttggaaaacagcggtgtcaccttttacaatacaaacggaaaacgccaggatatctttacaactttaaaacaggctggggtcaactatgttcgcgtccgcatctggaatcacccgtatgattcaaatggcaacgggtatggcgggggaaacaatgatgttcaaaaagccatcgaaatcggaaaaagagcgacagcgaacggaatgaaggtgctggccgactttcactactctgatttctgggccgatccagcgaaacaaaaggtgcccaaagcctgggcgaatctcagctttgaagcaaaaaaagcaaagctctatgagtatacgaaacaaagcctgcaaaagatgatcaaggaaggcgtgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 5 - DNA, 4254 bp, Artificial Sequence
  Integration cassette
gcaggctgttattgtaacatgtaagccataagccattcgtaaaagtgcgggaggaaggtcatgaataatctgcgtaatagactttcaggcgtgaatgggaaaaataagagagtaaaagaaaaagaacaaaaaatctggtcggagattgggatgatagcgggagcatttgcgctgcttgatgtgatcatccgcggcattatgtttgaatttccgtttaaagaatgggctgcaagccttgtgtttttgttcatcattatcttatattactgcatcagggctgcggcatccggaatgctcatgccgagaatagacaccaaagaagaactgcaaaaacgggtgaagcagcagcgaatagaatcaattgcggtcgcctttgcggtagtggtgcttacgatgtacgacagggggattccccatacattcttcgcttggctgaaaatgattcttctttttatcgtctgcggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaaatgtaagcgttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggccagtgaattcgagctcggtacctaccgttcgtataatgtatgctatacgaagttatgataaaaaatttagaagccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaatataacttcgtataatgtatgctatacgaacggtaggatcctctagagtcgacctgcaggcattttacatttttagaaatgggcgtgaaaaaaagcgcgcgattatgtaaaatataaagtgatagcggtaccattataggtaagagaggaatgtacacatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgcactcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaagcaatcaatgtcggatgccagcctggctttgattacgtgctaaatggtttatataatgactcgggcttaagcggttctcttccccattgagggcaaggctagacgggacttaccgaaagaaaccatcaatgatggtttcttttttgttcataaatcagacaaaacttttctcttgcaaaagtttgtgaagtgttgcacaatataaatgtgaaatacttcacaaacaaaaagacatcaaagagaaacataccctggaaggatgattaatgatgaacaaacatgtaaataaagtagctttaatcggagcgggttttgttggaagcagttatgcatttgcgttaattaaccaaggaatcacagatgagcttgtggtcattgatgtaaataaagaaaaagcaatgggcgatgtgatggatttaaaccacggaaaggcgtttgcgccacaaccggtcaaaacatcttacggaacatatgaagactgcaaggatgctgatattgtctgcatttgcgccggagcaaaccaaaaacctggtgagacacgccttgaattagtagaaaagaacttgaagattttcaaaggcatcgttagtgaagtcatggcgagcggatttgacggcattttcttagtcgcgacaaatccggttgatatcctgacttacgcaacatggaaattcagcggcctgccaaaagagcgggtgattggaagcggcacaacacttgattctgcgagattccgtttcatgctgagcgaatactttggcgcagcgcctcaaaacgtacacgcgcatattatcggagagcacggcgacacagagcttcctgtttggagccacgcgaatgtcggcggtgtgccggtcagtgaactcgttgagaaaaacgatgcg

(19) United States
(12) Patent Application Publication
(10) Pub. No.: US 2022/0290198 A1
(43) Pub. Date: Sep. 15, 2022

(54) Production of Sialylated Oligosaccharides in Bacillus Cells

(71) Applicant: Chr. Hansen HMO GmbH, Rheinbreitbach (DE)

(72) Inventors: Stefan Jennewein, Bad Honnef (DE); Dirk Wartenberg, Gau-Algesheim (DE); Stefanie Habersetzer, Bad Honnef (DE)

(73) Assignee: Chr. Hansen HMO GmbH, Rheinbreitbach (DE)

(21) Appl. No.: 17/639,729

(22) PCT Filed: Aug. 10, 2020
(86) PCT No.: PCT/EP2020/072429

§ 371 Date: Mar. 2, 2022

(30) Foreign Application Priority Data
Sep. 3, 2019 (EP) 19195148.2

(51) Int. Cl. C12P 19/18; A23L 33/00; C07H 3/06; C12P 19/26

(52) U.S. Cl. CPC C12P 19/18; A23L 33/40; C07H 3/06; C12P 19/26; C07H 1/08

(57) ABSTRACT

Disclosed are non-sporulating Bacillus cells for the production of a sialylated oligosaccharide wherein said Bacillus cell has been genetically engineered to possess a lactose permease, a CMP-NeuNAc biosynthesis pathway and a sialyltransferase, as well as a method for producing a sialylated oligosaccharide using said Bacillus cell.

The present invention relates to the technical field of genetic engineering, in particular to genetic engineering of Bacillus cells for the production of sialylated oligosaccharides in said Bacillus cells, to the fermentative production of sialylated oligosaccharides using said Bacillus cells, and to the use of thus produced sialylated oligosaccharides.

### BACKGROUND

Human milk provides young infants with all nutrients they need for healthy growth and development. The saccharides that are present in human milk represent its main component, ahead of fats and proteins. In addition to lactose, which serves as an energy source, human milk contains more complex saccharide molecules, i.e. oligosaccharides. Approximately 200 structurally distinct oligosaccharides have been identified in human milk to date. These oligosaccharides are found at significant concentrations only in human milk, and they are collectively known as human milk oligosaccharides (HMOs). Said HMOs are based on the disaccharide lactose (consisting of a glucose (Glc) moiety and a galactose (Gal) moiety) and bear additional monosaccharide residues which are based on N-acetyl-glucosamine (GlcNAc), fucose (Fuc), sialic acid/N-acetylneuraminic acid (NeuNAc), and/or galactose (Gal). The concentration and composition of HMOs in human milk varies between individuals and during the lactation period from up to 20 g/L in colostrum to 5-10 g/L in mature milk.

A considerable number of HMOs bear at least one NeuNAc moiety. Among these sialylated human milk oligosaccharides (SHMOs), 3′-sialyllactose, 6′-sialyllactose, sialyllacto-N-tetraose a, sialyllacto-N-tetraose b, sialyllacto-N-tetraose c and disialyllacto-N-tetraose are the most prevalent members in human milk.

Sialic acids (Sia) are a family of negatively charged monosaccharides with a nine-carbon backbone. More than 50 forms of these α-keto acids have been found in nature. The most abundant sialic acid appears to be N-acetylneuraminic acid (NANA, NeuNAc, Neu5Ac).

Sialic acids are present as terminal monosaccharide moieties of glycans that are present in glycoconjugates (glycoproteins and glycolipids) on the surface of cells of vertebrates and higher invertebrates. Sialic acids are components of the lipopolysaccharides and capsular polysaccharides of pathogenic bacteria including Escherichia coli K1, Haemophilus influenzae, Haemophilus ducreyi, Pateurella multocida, Neisseria gonorrhoeae, Neisseria meningitidis, Campylobacter jejuni and Streptococcus agalactiae.

Sialylated HMOs were observed to support the infant's resistance to enteropathogenic bacteria and viruses. Interestingly, recent studies further demonstrated a protective effect of long-chained SHMOs against necrotizing enterocolitis, which is one of the most common and lethal disease in preterm infants. Also, sialylated oligosaccharides have been shown to neutralize enterotoxins of various pathogenic microbes including Escherichia coli, Vibrio cholerae and Salmonella. Further, it was found that sialylated oligosaccharides interfere with the colonization of the gut by Helicobacter pylori and thereby prevent or inhibit gastric and duodenal ulcers. In addition, SHMOs are believed to support an infant's brain development and its cognitive capabilities.

Due to the known benefits of HMOs, especially of sialylated HMOs, an economically worthwhile process for their production is desired such that these sialylated oligosaccharides or at least some of these sialylated oligosaccharides become available as supplement for infant formulae.

The limited availability of human milk for other purposes than breast feeding an infant and the difficulties to obtain pure fractions of individual human milk oligosaccharides from natural sources led to the development of chemical routes for their synthesis. However, neither chemical synthesis nor biocatalytic in-vitro approaches proved to be commercially sustainable. Moreover, chemical synthesis of human milk oligosaccharides involves the use of several noxious chemicals which impose the risk of contaminating the final product.

As an alternative to chemical and biocatalytic in-vitro synthesis, fermentative production of HMOs has been developed. To date, recombinant Escherichia coli cells are used for microbial production of some HMOs in an industrial scale.

However, the genus Escherichia coli comprises pathogenic members as well as non-pathogenic members. Notwithstanding that non-pathogenic E. coli strains are employed for the microbial production of HMOs, such non-pathogenic E. coli are not recognized as safe for the manufacturing of products that are intended for human consumption in a variety of jurisdictions. This hinders regulatory approval of HMOs being manufactured by current biotechnological methods for human consumption in said jurisdictions. Therefore, microbial cells of genera that are recognized as safe for human consumption or recognized as safe when used in the production of compounds for human consumption in such jurisdictions are needed for the manufacturing saccharides that are intended for human consumption, e.g. human milk oligosaccharides, in particular for consumption by infants. The use of production strains recognized as safe reduces the—at least alleged—concerns with respect to the likelihood of the saccharide's risks for human health, and will facilitate their regulatory approval in most jurisdictions.

The problem is solved by using bacterial cells of the genus Bacillus for the production of sialylated oligosaccharides, in particular of sialylated HMOs. Bacterial cells of some species of the genus Bacillus are already recognized as being safe for human consumption or for the production of compounds/food for human consumption. Hence, provided are Bacillus cells of species and/or strains that are generally recognized as safe, for the production of sialylated oligosaccharides, in particular for the production of sialylated human milk oligosaccharides.

Bacteria of the genus Bacillus are gram-positive, rod-shaped, endospore-forming microbial cells of either aerobic or facultatively anaerobic species. The genus Bacillus belongs to the phylum Firmicutes. The genome of the members of the genus Bacillus possesses a bias towards A-T base pairs in its codon usage. Bacillus species are almost ubiquitously present in nature. They can for example be found in soil (B. subtilis), but also occur in extreme environments such as high pH (B. alcalophilus), high temperature (B. thermophilus), or high salt (B. halodurans).

The genus Bacillus includes 266 named species which include free living species as well as parasitic pathogenic species. Two Bacillus species are considered of being medically significant: B. anthracis, which causes anthrax, and B. cereus, which causes food poisoning. A third species, B. thuringiensis, is an important insect pathogen producing a toxin that can kill insects. Thus, it is used as an insecticide to control insect pests.

Because of their GRAS (Generally Recognized as Safe) status, several Bacillus species, e.g. B. amyloliquefaciens, B. licheniformis and B. subtilis, are used in the biotechnological production of various proteins and compounds to be used in food and pharmaceutical industry.

Bacillus amyloliquefaciens is the source of restriction enzyme BamHI, and also synthesizes the natural antibiotic protein barnase. In addition, B. amyloliquefaciens produces plantazolicin, an antibiotic with selective activity against B. anthracis. Alpha-amylase from B. amyloliquefaciens is often used in starch hydrolysis. B. amyloliquefaciens is also a source of subtilisin, which catalyzes the breakdown of proteins.

Bacillus amyloliquefaciens is a root-colonizing bacterium that is used to fight some plant root pathogens in agriculture, aquaculture and hydroponics as it acts against bacterial and fungal pathogens, and may prevent infection by competitive exclusion or out-competing the unwanted pathogen.

Its high capacity of secreting alkaline serine protease has made B. licheniformis one of the most important bacteria in industrial enzyme production. Subtilisin Carlsberg secreted by B. licheniformis is used as a detergent protease, and is sold under the trade name Alcalase®.

Bacillus subtilis is a catalase positive bacterium that is found in soil and the gastrointestinal tract of ruminants and humans. B. subtilis and substances derived from this endotoxin-free bacterium have been evaluated by different authoritative bodies for their safe and beneficial use in food. In the United States carbohydrase and protease enzymes from B. subtilis are recognized as generally safe (GRAS) by the US Food and Safety Administration (FDA). Bacillus subtilis has also been granted the “Qualified Presumption of Safety” status by the European Food Safety Authority.

Moreover, nontoxigenic and nonpathogenic strains of B. subtilis strain are commonly used in food. For example, fermented soy beans in the form of Natto are frequently consumed in Japan and contain as many as 10⁸ viable B. subtilis cells per gram. Natto is recognized for its contribution to a healthy gut flora and vitamin K₂ intake.

The Natto product and the B. subtilis var. natto as its principal component are FOSHU (Foods for Specified Health Use) approved by the Japanese Ministry of Health, Labor and Welfare as effective for preservation of health.

Bacillus subtilis is easy to handle, grows rapidly and the culture conditions are simple. Recombinant B. subtilis strains are used in the production of polyhydroxyalkanolates, hyaluronic acid and various enzymes such as amylase and proteases.

Wild-type natural isolates of B. subtilis are difficult to work with as compared to laboratory strains that have undergone domestication processes of mutagenesis and selection. These domesticated strains often have improved capabilities to undergo natural competence development (uptake and integration of environmental DNA), improved capabilities of growth and loss of abilities needed “in the wild”. In B. subtilis, linear DNA as well as multimeric forms of plasmid DNA are actively taken up by natural competent cells.

Under certain physiological conditions, a small subpopulation of B. subtilis cells becomes competent. In B. subtilis, natural competence is regulated by a complex regulatory network. Key regulators in this network are, amongst others, the master regulator of competence ComK and the transcriptional master regulator of sporulation Spo0A. Transformation efficiency of B. subtilis cells and probably the efficacy for integrating DNA into their genome can be improved by genetic engineering. This can be achieved by ectopic integration of an expression cassette, comprising a regulated promoter (e.g. the mannitol-inducible P_{mtlA} promoter) and the genes comK and comS, into the genome of B. subtilis. Additionally, this strategy allows transformation of B. subtilis by natural competence using a complex medium (e.g. LB).

For the production of sialylated oligosaccharides, Bacillus cells can be genetically engineered by various methods.

The term “genetically engineered” as used herein refers to the modification of the Bacillus cell's genetic make-up using molecular biological methods. The modification of the Bacillus cell's genetic make-up may include the transfer of genes within and/or across species boundaries, inserting, deleting, replacing and/or modifying nucleotides, triplets, genes, open reading frames, promoters, enhancers, terminators and other nucleotide sequences mediating and/or controlling gene expression. The modification of the Bacillus cell's genetic make-up aims to generate a genetically modified Bacillus cell possessing particular, desired properties. Genetically engineered Bacillus cells can contain one or more genes that are not present in the native (not genetically engineered) form of the cell. Techniques for introducing exogenous nucleic acid molecules and/or inserting exogenous nucleic acid molecules (recombinant, heterologous) into a Bacillus cell's hereditary information for inserting, deleting or altering the nucleotide sequence of a cell's genetic information are known to the skilled artisan. Genetically engineered Bacillus cells can contain one or more genes that are present in the native form of the cell, wherein said genes are modified and re-introduced into the Bacillus cell by artificial means. The term “genetically engineered” also encompasses Bacillus cells that contain a nucleic acid molecule being endogenous to the cell, and that has been modified without removing the nucleic acid molecule from the cell. Such modifications include those obtained by gene replacement, site-specific mutations, and related techniques.

Gene integration and/or (simultaneous) gene inactivation by disruption or deletion can be achieved by homologous recombination. For an efficient homologous recombination, at least 400-500 bp of homology arms are necessary in B. subtilis.

Another method for targeted genome engineering is the CRISPR-Cas9 system. This rapid and markerless genome-editing tool can be used for large scale genomic deletions, small and large DNA insertions, gene silencing by introduction of a stop codon as well as introduction of point mutations. No previous genome modifications are required for the scarless genome editing by CRISPR-Cas9.

Random chromosomal integration of genes and insertional mutagenesis can be performed using a modified mariner-derived transposon. This system is not biased towards hotpots in B. subtilis while showing high efficiency in random ectopic integration.

Although Bacillus species are used for industrial scale production of enzymes, Bacillus cells have not been implemented to date for industrial scale production of oligosaccharides, in particular of industrial scale production of sialylated oligosaccharides.

Chinese patent application CN 108 410 787 A discloses recombinant Bacillus subtilis cells which synthesize lactyl-N-neotetraose. Said recombinant B. subtilis cells possess a lactose permease gene which has been integrated into the cell's genome. In addition, said Bacillus cell which bears a plasmid comprising a β-1,3-N-glucosamine transferase gene and a β-1,4-galactosyltransferase gene. The B. subtilis cells may be cultivated in the presence of exogenous lactose, and synthesizes lactyl-N-neotetraose at titers of up to about 1 g/L which is too little for economically reasonable industrial scale production.

Chinese patent application CN 109 735 479 A discloses recombinant Bacillus subtilis cells for synthesizing 2′-fucosyllactose, wherein the expression of a lactose transport enzyme is enhanced, and which cell expresses a fucose kinase, a phosphate guanine transferase and a fucosyltransferase. The yield of 2′-fucosyllactose in the fermentation medium was reported to be between 0.424 g/L and 1.042 g/L.

Although a variety of patent applications mention Bacillus as a genus which is deemed to be suitable for the production of neutral oligosaccharides such as lacto-N-neotetraose or 2′-fucosyllactose, use of Bacillus for the productions of sialylated oligosaccharides, in particular of sialylated human milk oligosaccharides, has not been implemented yet, presumably because of the significant efforts in metabolic engineering that are required to implement the necessary biosynthetic pathways for HMO production in Bacillus. Whereas above-referenced B. subtilis for the production of LNnT relies on donor substrates that are naturally occurring in B. subtilis cells, production of a sialylated oligosaccharide in Bacillus requires implementation of a heterologous metabolic pathway in the cell for providing a donor substrate, i.e. CMP-NeuNAc, that is essential for the synthesis of sialylated oligosaccharides.

The object has been achieved by providing a Bacillus cell that possesses a lactose permease for import of exogenous lactose into the cell, a CMP-NeuNAc biosynthesis pathway for the intracellular formation of a nucleotide-activated sialic acid, i.e. cytidine monophosphate N-acetylneuraminic acid (CMP-NeuNAc), as donor substrate for a sialic acid moiety, and a sialyltransferase for transfer of the sialic acid moiety from CMP-NeuNAc to an acceptor substrate. Cultivation of such Bacillus cells in the presence of exogenous lactose allows production of a desired sialylated oligosaccharide.

### SUMMARY

According to a first aspect, provided is a non-sporulating bacterial cell of the genus Bacillus for the production of a sialylated oligosaccharide, wherein the Bacillus cell possesses a lactose permease, a CMP-NeuNAc biosynthesis pathway and a sialyltransferase.

According to a second aspect, provided is the use of the non-sporulating bacterial cell of the genus Bacillus according to the first aspect for the production of a sialylated oligosaccharide.

According to a third aspect, provided is a method for the production of a sialylated oligosaccharide, the method comprising:
providing a non-sporulating bacterial cell of the genus Bacillus, wherein said Bacillus cell possesses a lactose permease, a CMP-NeuNAc biosynthesis pathway and a sialyltransferase;
cultivating the Bacillus cell in a culture medium containing lactose and under conditions that are permissive for the Bacillus cell to produce the sialylated oligosaccharide, and
optionally retrieving the sialylated oligosaccharide from the culture medium and/or the Bacillus cell.

According to a fourth aspect, provided are sialylated oligosaccharides that were produced by a Bacillus cell which possesses a lactose permease, a CMP-NeuNAc biosynthesis pathway and a sialyltransferase.

According to a fifth aspect, provided is the use of a sialylated oligosaccharide that was produced by a Bacillus cell which possesses a lactose permease, a CMP-NeuNAc biosynthesis pathway and a sialyltransferase for the manufacturing of a nutritional composition.

According to a sixth aspect, provided are nutritional compositions containing at least one sialylated oligosaccharide that had been produced by a Bacillus cell which possesses a lactose permease, a CMP-NeuNAc biosynthesis pathway and a sialyltransferase.

### DESCRIPTION OF THE FIGURES

FIG. 1 illustrates different embodiments of the CMP-NeuNAc biosynthesis pathway that is implemented in a Bacillus cell for the production of a sialylated oligosaccharide.

### DETAILED DESCRIPTION

According to the first aspect, provided is a non-sporulating bacterial cell of the genus Bacillus for the production of a sialylated oligosaccharide. For being able to produce a sialylated oligosaccharide, the Bacillus cell has to provide a donor substrate comprising a sialic acid moiety, and an acceptor substrate being a disaccharide or an oligosaccharide to a sialyltransferase such that the sialyltransferase can transfer the sialic acid moiety from the donor substrate to said acceptor substrate thereby generating the sialylated oligosaccharide.

It is to be understood that the sialylated oligosaccharide said Bacillus cell is intended to produce is the desired sialylated oligosaccharide, whereas other sialylated oligosaccharides that may be generated due to the promiscuity of the sialyltransferase during production of the desired sialylated oligosaccharide in the Bacillus cell are considered as undesired sialylated oligosaccharides or by-products.

The Bacillus cell for producing a sialylated oligosaccharide is a non-sporulating Bacillus cell. Wild-type Bacillus cells are capable of forming spores. Sporulation, i.e. the process of forming spores, in bacteria is considered to be a reaction of the bacterial cell which initiates a developmental program leading to the formation of daughter cells of distinct morphology and fate. Sporulation of Bacillus was studied as a basic model for cell differentiation. During sporulation, a rod-shaped Bacillus cell divides asymmetrically, thereby resulting in two genetically identical cells with different morphology and fates.

However, in industrial production it is not desired if the bacterial production strain sporulates during fermentative production of the desired product. Therefore, it is preferred to use Bacillus cells for the production of sialylated oligosaccharides that are not capable of forming spores. Such Bacillus cells are called “non-sporulating”.

In an embodiment, the non-sporulating Bacillus cell for the production of a sialylated oligosaccharide originated from one of the B. subtilis strains listed in Table 1.

TABLE 1
Non-limiting list of Bacillus subtilis strains.

| Strain | Manufacturer/Reference | Product | Application |
|---|---|---|---|
| Bacillus subtilis 168 | BGSCID 1A1 | — | reference strain |
| Bacillus subtilis 168 | BGSCID BKE15310 | — | reference strain |
| ΔspollGA::erm | | | |
| Bacillus subtilis 168 | BGSCID BKE15320 | — | reference strain |
| ΔsigE::erm | | | |
| Bacillus subtilis168 | BGSCID BKE15330 | — | reference strain |
| ΔsigG::erm | | | |
| Bacillus subtilis 168 | BGSCID BKE23450 | — | reference strain |
| ΔsigF::erm | | | |
| Bacillus subtilis 3NA | BGSCID 1S1 | — | reference strain |
| Bacillus subtilis W23 | BGSCID 2A9 | — | reference strain |
| Bacillus subtilis PY79 | BGSCID 1A747 | — | reference strain |
| Bacillus subtilis | Garden of Life ® | Primal Defense ® | Human use |
| Bacillus subtilis | Roux-Ocefa Laboratorios | Totalflora | Human use |
| Bacillus subtilis | lst. Bioch. Italiano | Lactipan Plus | Human use |
| Bacillus subtilis 2335 | Biopharma | Biosporin ® | Human use |
| LifeinU ™ Bacillus subtilis | Lesaffre Human Care | Super Smart ® | Human use |
| CU1 | | | |
| Bacillus subtilis PXN/21 | Probiotics International Ltd | Bio-Kult ® | Human use |
| Bacillus subtilis HU58 | Microbiome LABS | HU58 ™ | Human use |
| Bacillus subtilis R0179 | Hanmi Pharmaceutical Co. | Medilac ® | Human use |
| | Ltd. | | |
| Bacillus subtilis DE111 | Deerland Enzymes & | NutriCommit | Human use |
| | Probiotics | | |
| Bacillus subtilis DSM21097 | MorinagaGarden B.V. | Morinaga Plus⁺ | Human use |
| Bacillus subtilis IDCC1101 | Ildong Pharmaceutical | Biobaby ® | Human use |
| Bacillus subtilis | JSL Foods | YoguFarm | Human use |
| KATMIRA1933 | | | |
| Bacillus subtilis spp. Natto | various | Natto | Human use |
| Bacillus subtilis SC-8 | various | Cheonggukjang | Human use |
| Bacillus subtilis | Christian Hansen | BioPlus2B ® | Veterinary Use |
| CH201/DSM5750 | | | |
| Bacillus subtilis PB6 | Kenim ® | CLOSTAT ® | Veterinary Use |

In some embodiments, the Bacillus cell has been genetically engineered to become non-sporulating, for example by deletion or functional inactivation of Spo0A. Spo0A is a DNA-binding protein which influences, directly or indirectly, the expression of more than 500 genes during the early stage of sporulation. Suitable functional inactivation of Spo0A includes deletion of the phosphorylation site at which Spo0F and Spo0B phosphotransferases phosphorylate Spo0A. Deletion or functional inactivation of the Spo0F and Spo0B phosphotransferase phosphorylation site leads to a functional inactivation of Spo0A and thereby impairs the Bacillus cell's ability to sporulate. Alternatively, the gene or nucleotide sequence encoding Spo0A, or part of the gene or nucleotide sequence encoding Spo0A may be deleted from the Bacillus cell's genome.

In an additional and/or alternative embodiment, the Bacillus cell has been genetically engineered by deletion or functional inactivation of the gene(s) encoding sigma factor SigE (sigE) and/or sigma factor SigF (sigF). Sigma factors SigE and SigF are transcription factors that are involved in the expression of genes which encode proteins that are involved in the early phase of sporulation.

Deletion or functional inactivation of Spo0A, SigE and/or SigF impairs the sporulation ability of a Bacillus cell to sporulate. Such non-sporulating Bacillus cell may be used as a progenitor for generating a Bacillus cell that is able to produce a sialylated oligosaccharide.

Wild-type Bacillus cells neither synthesize lactose intracellularly nor do they internalize exogenous lactose. However, lactose is the acceptor substrate for a sialic acid moiety by a lactose-accepting sialyltransferase in the formation of some sialylated oligosaccharides such as 3′-sialyllactose (3-SL) or 6′-sialyllactose (6-SL). Hence, to be able to produce such sialylated oligosaccharides, the Bacillus cell has to have the capability of providing lactose to the lactose-accepting sialyltransferase, either by generating lactose intracellularly and/or by internalizing exogenous lactose. Moreover, lactose is typically the disaccharide moiety most if—not all—sialylated HMOs bear. Hence, internalization of lactose is also required for the biosynthesis of other sialylated HMOs than trisaccharides, i.e. when the acceptor substrate for the NeuNAc moiety is an oligosaccharide rather than the disaccharide lactose.

In an embodiment, the Bacillus cell for producing a sialylated oligosaccharide possesses a lactose permease for internalization of exogenous lactose. Hence, the Bacillus cell is able to internalize exogenous lactose. Alternatively, the Bacillus cell may be genetically engineered to synthesize lactose intracellularly from glucose and galactose such that an exogenous supply of lactose will not be necessary for the production of a sialylated oligosaccharide.

The term “exogenous” with respect to lactose as used herein refers to lactose that does not originate from the Bacillus cell for the production of the sialylated oligosaccharide, i.e. being intracellularly synthesized by said Bacillus cell, but which originates from outside of said Bacillus cell and which is added to the culture medium in which the Bacillus cell for producing the sialylated oligosaccharide is grown to produce the sialylated oligosaccharide.

In some embodiments, the Bacillus cell has been genetically engineered to be able to internalize exogenous lactose, i.e. it has been genetically engineered to possess a lactose permease. Thus, the Bacillus cell for producing a sialylated oligosaccharide possesses a heterologous lactose permease. A suitable heterologous lactose permease is E. coli LacY or a functional variant thereof.

The term “heterologous” as used herein with respect to proteins, polypeptides, enzymes and transporters, but also with respect to nucleic acid molecules, nucleotide sequences and/or genes, refers to a molecule that is non-native to the species of the Bacillus cell which contains said molecule. The term “non-native” indicates that said molecule is not present in the naturally occurring or wild-type progenitor Bacillus cell, i.e. in the Bacillus cell of the same species that is most commonly occurring in nature. Thus, a “heterologous sequence” or a “heterologous nucleic acid” or “heterologous polypeptide”, as used herein, is one that originates from a source foreign to the Bacillus cell (e.g. from a different species), or, if from the same source, is modified from its original form. The heterologous sequence may be stably introduced, e.g. by transfection, transformation, conjugation or transduction, into the genome of the host microbial host cell, thus representing a genetically modified host cell. Techniques may be applied which will depend on the host cell the sequence is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989). Accordingly, a “heterologous polypeptide” is a polypeptide that does not naturally occur in the cell, and a “heterologous sialyltransferase” is a sialyltransferase that does not naturally occur in the microbial cell.

The term “functional variant” as used herein with respect to enzymes and/or transport molecules refers to proteins or polypeptides possessing the same activity (enzymatic, catalytic or translocating) as the referenced enzyme or transporter, but which has a different amino acid sequence than the referenced enzyme or transporter molecule. Thus, a typical variant of a protein/polypeptide differs in amino acid sequence from the reference protein/polypeptide. A variant and the reference protein/polypeptide may differ in amino acid sequence by one or more substitutions, additions, and/or deletions in any combination. Hence, the term “functional variant” comprises truncated versions of the referenced protein/polypeptide which possesses the same activity as the reference protein/polypeptide. A substituted or inserted amino acid residue may or may not be one encoded by the genetic code. A variant of a protein/polypeptide may be naturally occurring such as an allelic variant, or it may be a variant that is not known to occur naturally. Non-naturally occurring variants of proteins/polypeptides may be made by mutagenesis techniques, by direct synthesis, and by other recombinant methods known to the persons skilled in the art. Within the scope of the present disclosure, also proteins and interspecies homologs are comprised by the term “variant” that have an amino acid sequence that has greater than about 60% amino acid sequence identity, preferably 65%, 70%, 75%, 80%, 85%, 90%, more preferably 91%, 92%, 93%, 94%, 95%, 96%, 97%, 98% or 99% or greater amino acid sequence identity, preferably over a region of at least about 25, 50, 100, 200, 500, 1000, or more amino acids, to the referenced polypeptide.

The term “same activity” as used herein refers to an enzymatic, catalytic or transporting activity of a protein/polypeptide in qualitative manner only. Hence, “functional variant” also comprises variants which possess an increased or decreased activity as compared to the activity of the referenced protein/polypeptide.

In various embodiments, the Bacillus cell has been genetically engineered to contain and express a nucleotide sequence which encodes a lactose permease, preferably a nucleotide sequence that encodes the E. coli lactose permease LacY (UniProtKB—P02920) or a functional variant thereof.

The E. coli lactose permease LacY is encoded by the protein coding region (i.e. the open reading frame) of the E. coli lacY gene (GenBank acc. no: NP_414877.1).

Hence, in some embodiments, the Bacillus cell has been genetically engineered to contain and express a nucleotide sequence encoding the protein-coding region of E. coli lacY.

In some embodiments, the codon usage of the nucleotide sequence encoding the heterologous lactose permease is adapted to the codon usage of Bacillus. The codon usage of B. subtilis for example is peculiar in that the overall GC-content is below about 45%, the GC-content of the first letter of the codons above about 51%, the CG-content of the second letter of the codons below about 36.1%, and the CG-content of the third letter of the codons below about 46%.

For expression of the lactose permease, the Bacillus cell contains a recombinant lactose permease gene, wherein the nucleotide sequence which encodes the lactose permease is operably linked to expression control sequences which mediate expression of the nucleotide sequence encoding the lactose permease.

The term “operably linked” as used herein shall mean a functional linkage between a nucleotide sequence which encodes a polypeptide or protein (typically referred to as “protein coding region”, “open reading frame”, and sometimes even as “gene”) and a second nucleotide sequence (the expression control sequence such as a promoter, signal sequence, or array of transcription factor binding sites) which effects transcription and/or translation of the nucleotide sequence which encodes the polypeptide or protein. Accordingly, the term “promoter” designates deoxyribonucleic acid (DNA) sequences which usually “precede” an open reading frame in a DNA polymer and provides a site for initiation of the transcription into mRNA. “Regulator” DNA sequences, also usually “upstream” of (i.e., preceding) an open reading frame in a given DNA polymer, bind proteins that determine the frequency (or rate) of transcriptional initiation. Collectively referred to as “promoter/regulator” or “control” DNA sequence, these sequences which precede a selected open reading frame (or series of open reading frames) in a functional DNA polymer cooperate to determine whether the transcription (and eventual expression) of an open reading frame will occur. DNA sequences which “follow” a gene in a DNA polymer and provide a signal for termination of the transcription into mRNA are referred to as transcription “terminator” sequences.

The recombinant lactose permease gene may be present as being integrated into the Bacillus chromosome, or present as an episomal version on an additional plasmid within the Bacillus cell.

Expression of the heterologous lactose permease gene in the Bacillus cell enables the resulting Bacillus cell to internalize exogenously supplied lactose from the culture medium. The internalized lactose can then serve as an acceptor substrate for a sialic acid moiety to be transferred by a lactose-accepting sialyltransferase (see herein below), for example in the formation of 3′-SL or 6′-SL.

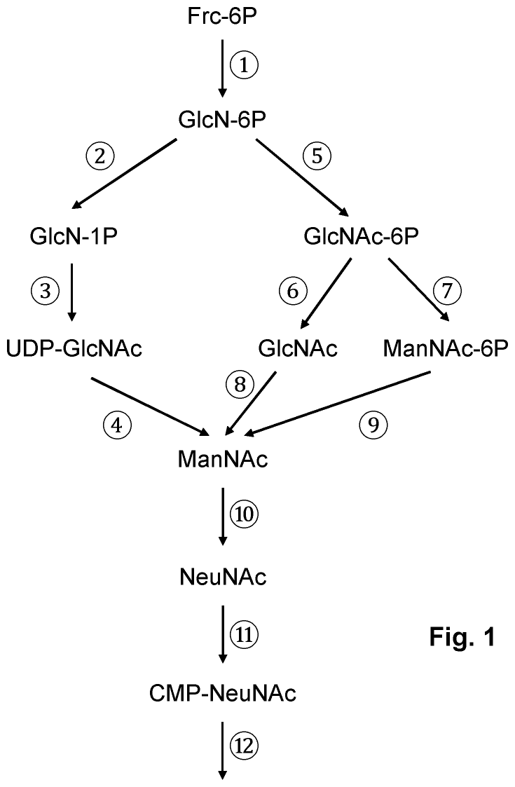

For the production of a sialylated oligosaccharide, the Bacillus has to be able to provide a donor substrate for transferring a NeuNAc moiety to an acceptor substrate. An exemplary donor substrate for a NeuNAc moiety is CMP-NeuNAc. Hence, the Bacillus cell has to be able to intracellularly produce CMP-NeuNAc for the production of sialylated oligosaccharides. For intracellular biosynthesis of CMP-NeuNAc, the Bacillus cell possesses a CMP-NeuNAc biosynthesis pathway (FIG. 1). Thus, the Bacillus cell has been genetically engineered to possess a CMP-NeuNAc biosynthesis pathway.

The CMP-NeuNAc biosynthesis pathway comprises a NeuNAc salvage pathway or a sialic acid biosynthesis pathway for intracellular de novo biosynthesis of NeuNAc. Hence, the Bacillus cell possesses a CMP-NeuNAc biosynthesis pathway comprising a NeuNAc salvage pathway and/or a sialic acid biosynthesis pathway.

The NeuNAc salvage pathway comprise internalization of exogenous sialic acid by the Bacillus cell, and conversion of the internalized sialic acid to CMP-NeuNAc. For internalization of exogenous NeuNAc, the Bacillus cell possesses a sialic acid transporter. In some embodiments, the genetically engineered Bacillus cell has been genetically engineered to possess a sialic acid transport for internalization of exogenous NeuNAc. A suitable sialic acid transporter is E. coli NanT (UniProtKB P41036). E. coli NanT catalyzes the proton-dependent symport of sialic acid. NanT can transport NeuNAc as well as the related sialic acids N-glycolylneuraminic acid (NeuNGc) and 3-keto-3-deoxy-D-glycero-D-galactonononic acid (KDN). In an embodiment, the genetically engineered Bacillus cell possesses E. coli NanT or a functional variant thereof.

In some embodiments, the Bacillus cell has been genetically engineered to contain and express a nucleotide sequence which encodes a sialic acid transporter for internalization of exogenous NeuNAc, preferably a nucleotide sequence that encodes E. coli NanT or a functional variant thereof.

The E. coli NanT is encoded by the protein-coding region of the E. coli nanT gene. Hence, in some embodiments, the Bacillus cell has been genetically engineered to contain and express a nucleotide sequence that encodes the E. coli sialic acid transporter NanT or a functional variant thereof.

For expression of the sialic acid transporter, the Bacillus cell contains a recombinant sialic acid transporter gene, wherein the nucleotide sequence that encodes the sialic acid transporter is operably linked to expression control sequences which mediate expression of the nucleotide sequence encoding the sialic acid transporter.

The recombinant sialic acid transporter gene may be present as being integrated into the Bacillus chromosome, or present as an episomal version on an additional plasmid in the Bacillus cell.

In some embodiment, the codon usage of the nucleotide sequence encoding the sialic acid transporter is adapted to the codon usage of Bacillus.

In various embodiments, the CMP-NeuNAc biosynthesis pathway comprises a sialic acid biosynthesis pathway. Thus, the Bacillus cell may comprise a sialic acid biosynthesis pathway for the intracellular biosynthesis of N-acetylneuraminic acid.

The sialic acid biosynthesis pathway comprises the enzymatic activities of a glutamine:fructose-6-phosphate aminotransferase (FIG. 1: \textcircled{1}) and an N-acetylneuraminic acid synthase (=sialic acid synthase) (FIG. 1: \textcircled{10}). Hence, the Bacillus cell for the production of a sialylated oligosaccharide possesses a glutamine:fructose-6-phosphate aminotransferase and an N-acetylneuraminic acid synthase.

The enzyme glutamine:fructose-6-phosphate aminotransferase (EC 2.6.1.16) catalyzes the conversion of fructose-6-phosphate (Frc-6P) to glucosamine-6-phosphate (GlcN-6P) using glutamine (FIG. 1: \textcircled{1}). This enzymatic reaction is typically considered to be the first step in the hexosamine biosynthesis pathway. Alternative names of the glutamine:fructose-6-phosphate aminotransferase are D-fructose-6-phosphate aminotransferase, GFAT, glucosamine-6-phosphate synthase, hexosephosphate aminotransferase, and L-glutamine-D-fructose-6-phosphate aminotransferase.

In an additional and/or alternative embodiment, the genetically engineered Bacillus cell possesses a glutamine:fructose-6-phosphate aminotransferase, preferably a heterologous a glutamine:fructose-6-phosphate aminotransferase. An example of a suitable heterologous glutamine:fructose-6-phosphate aminotransferase is derived from E. coli. The E. coli glutamine:fructose-6-phosphate aminotransferase (UniProtKB—P17169) is designated GlmS. Hence, the Bacillus cell possesses an E. coli GlmS or a functional variant of the E. coli GlmS. Preferably, the functional variant of the E. coli GlmS is a version which shows significantly reduced sensitivity to glucosamine-6-phosphate inhibition as compared to the wild-type enzyme. An example of a functional variant of the E. coli GlmS which shows significantly reduced sensitivity to glucosamine-6-phosphate inhibition. Examples of functional variants of the E. coli GlmS which shows significantly reduced sensitivity to glucosamine-6-phosphate inhibition are GlmS*54 and GlmS* as described in international application No. PCT/EP2019/063669 (incorporated herein by reference).

In an additional and/or alternative embodiment, the genetically engineered Bacillus cell contains a nucleic acid molecule comprising a nucleotide sequence which encodes a glutamine:fructose-6-phosphate aminotransferase, preferably the E. coli glutamine:fructose-6-phosphate aminotransferase GlmS, or a nucleotide sequence encoding a functional variant of the E. coli GlmS which shows significantly reduced sensitivity to glucosamine-6-phosphate inhibition as compared to the wild-type enzyme.

For expression of the glutamine:fructose-6-phosphate aminotransferase, the Bacillus cell contains a recombinant glutamine:fructose-6-phosphate aminotransferase gene, wherein the nucleotide sequence which encodes the glutamine:fructose-6-phosphate aminotransferase is operably linked to expression control sequences which mediate expression of the glutamine:fructose-6-phosphate aminotransferase open reading frame.

In an additional and/or alternative embodiment, the codon usage of the nucleotide sequence encoding the heterologous glutamine:fructose-6-phosphate aminotransferase is adapted to the codon usage of Bacillus.

The recombinant glutamine:fructose-6-phosphate aminotransferase gene may be integrated into the Bacillus chromosome, or present as an episomal version on a plasmid within the Bacillus cell.

In an additional and/or alternative embodiment, the genetically engineered Bacillus cell comprises a sialic acid synthase. The sialic acid synthase catalyzes the condensation of N-acetylmannosamine (ManNAc) and phosphoenolpyruvate (PEP) to N-acetylneuraminic acid (NeuNAc) (FIG. 1: \textcircled{10}). The enzymatic formation of NeuNAc is the last step of the sialic acid biosynthesis pathway.

In an additional and/or alternative embodiment, the genetically engineered Bacillus cell comprises a sialic acid synthase or a functional variant thereof, preferably a heterologous sialic acid synthase. Examples of sialic acid synthases are known from a variety of bacterial species such as Campylobacter jejuni, Streptococcus agalactiae, Butyrivibrio proteoclasticus, Methanobrevibacter ruminantium, Acetobacterium woodii, Desulfobacula toluolica, Escherichia coli, Prevotella nigescens, Halorhabdus tiamatea, Desulfotignum phosphitoxidans, or Candidatus Scalindua sp., Idomarina loihiensis, Fusobacterium nucleatum or Neisseria meningitidis. An example of a suitable sialic acid synthase is the N-acetylneuraminic acid synthase NeuB of C. jejuni as encoded by the C. jejuni neuB gene.

Thus, in an additional and/or alternative embodiment, the genetically engineered Bacillus cell contains a nucleic acid molecule comprising and expressing a nucleotide sequence which encodes a N-acetylneuraminic acid synthase. Hence, the nucleotide sequence which encodes a N-acetylneuraminic acid synthase is operably linked to at least one nucleic acid expression control sequence effecting transcription and/or translation of said nucleotide sequence which encodes a N-acetylneuraminic acid synthase in the genetically engineered Bacillus cell to provide intracellular N-acetylneuraminic acid synthase activity.

In an additional and/or alternative embodiment, the codon usage of the nucleotide sequence encoding the heterologous N-acetylneuraminic acid synthase is adapted to the codon usage of Bacillus.

The recombinant N-acetylneuraminic acid synthase gene may be integrated into the Bacillus chromosome, or present as an episomal version on a plasmid within the Bacillus cell.

In an embodiment, the sialic acid biosynthesis pathway comprises uridine diphosphate-N-acetylglucosamine (UDP-GlcNAc) as an intermediate compound. The Bacillus cell comprising a sialic acid biosynthesis pathway which comprises UDP-GlcNAc as intermediate compound further possesses a phosphoglucosamine mutase (FIG. 1: \textcircled{2}), a glucosamine-1-phosphate N-acetyltransferase (FIG. 1: \textcircled{3}), an N-acetylglucosamine-1-phosphate uridyltransferase (FIG. 1 \textcircled{4}), and a UDP-N-acetylglucosamine 2-epimerase (FIG. 1: \textcircled{4}).

The phosphoglucosamine mutase converts glucosamine-6-phosphate (GlcN-6P) to glucosamine-1-phosphate (GlcN-1P).

In an additional and/or alternative embodiment, the Bacillus cell has been genetically engineered to possess a phosphoglucosamine mutase. A suitable phosphoglucosamine mutase is the E. coli phosphoglucosamine mutase GlmM or a functional variant thereof. Said E. coli GlmM is encoded by the E. coli glmM gene.

In an additional and/or alternative embodiment, the Bacillus cell has been genetically engineered to contain and express a nucleotide sequence which encodes a phosphoglucosamine mutase, preferably a nucleotide sequence that encodes the E. coli GlmM or a functional variant thereof.

The E. coli phosphoglucosamine mutase GlmM is encoded by the protein coding region of the E. coli glmM gene. Hence, the Bacillus cell has been genetically engineered to contain and express a nucleotide sequence that encodes the E. coli GlmM.

Thus, in an additional and/or alternative embodiment, the genetically engineered Bacillus cell contains a nucleic acid molecule comprising and expressing a nucleotide sequence which encodes a phosphoglucosamine mutase. Hence, the nucleotide sequence which encodes a phosphoglucosamine mutase is operably linked to at least one nucleic acid expression control sequence effecting transcription and/or translation of said nucleotide sequence which encodes a phosphoglucosamine mutase in the genetically engineered Bacillus cell to provide intracellular phosphoglucosamine mutase activity.

In an additional and/or alternative embodiment, the codon usage of the nucleotide sequence encoding the heterologous phosphoglucosamine mutase is adapted to the codon usage of Bacillus.

The recombinant phosphoglucosamine mutase gene may be integrated into the Bacillus chromosome, or present as an episomal version on a plasmid within the Bacillus cell.

The glucosamine-1-phosphate N-acetyltransferase catalyzes the transfer of an acetyl group from acetyl coenzyme A to glucosamine-1-phosphate (GlcN-1-P) to produce N-acetylglucosamine-1-phosphate (GlcNAc-1-P).

In an additional and/or alternative embodiment, the Bacillus cell has been genetically engineered to possess a glucosamine-1-phosphate N-acetyltransferase.

In an additional and/or alternative embodiment, the Bacillus cell has been genetically engineered to contain and express a nucleotide sequence which encodes a glucosamine-1-phosphate N-acetyltransferase.

Thus, in an additional and/or alternative embodiment, the genetically engineered Bacillus cell contains a nucleic acid molecule comprising and expressing a nucleotide sequence which encodes a glucosamine-1-phosphate N-acetyltransferase. Hence, the nucleotide sequence which encodes a glucosamine-1-phosphate N-acetyltransferase is operably linked to at least one nucleic acid expression control sequence effecting transcription and/or translation of said nucleotide sequence which encodes a glucosamine-1-phosphate N-acetyltransferase in the genetically engineered Bacillus cell to provide intracellular glucosamine-1-phosphate N-acetyltransferase activity.

In an additional and/or alternative embodiment, the codon usage of the nucleotide sequence encoding the heterologous glucosamine-1-phosphate N-acetyltransferase is adapted to the codon usage of Bacillus.

The recombinant glucosamine-1-phosphate N-acetyltransferase gene may be integrated into the Bacillus chromosome, or present as an episomal version on a plasmid within the Bacillus cell.

N-acetylglucosamine-1-phosphate uridyltransferase converts N-acetylglucosamine-1-phosphate (GlcNAc-1-P) into UDP-GlcNAc by the transfer of uridine 5-monophosphate (from uridine 5-triphosphate).

In an additional and/or alternative embodiment, the Bacillus cell has been genetically engineered to possess a N-acetylglucosamine-1-phosphate uridyltransferase.

In an additional and/or alternative embodiment, the Bacillus cell has been genetically engineered to contain and express a nucleotide sequence which encodes a N-acetylglucosamine-1-phosphate uridyltransferase.

Thus, in an additional and/or alternative embodiment, the genetically engineered Bacillus cell contains a nucleic acid molecule comprising and expressing a nucleotide sequence which encodes a N-acetylglucosamine-1-phosphate uridyltransferase. Hence, the nucleotide sequence which encodes a N-acetylglucosamine-1-phosphate uridyltransferase is operably linked to at least one nucleic acid expression control sequence effecting transcription and/or translation of said nucleotide sequence which encodes a N-acetylglucosamine-1-phosphate uridyltransferase in the genetically engineered Bacillus cell to provide intracellular N-acetylglucosamine-1-phosphate uridyltransferase activity.

In an additional and/or alternative embodiment, the codon usage of the nucleotide sequence encoding the heterologous N-acetylglucosamine-1-phosphate uridyltransferase is adapted to the codon usage of Bacillus.

The recombinant N-acetylglucosamine-1-phosphate uridyltransferase gene may be integrated into the Bacillus chromosome, or present as an episomal version on a plasmid within the Bacillus cell.

In an additional and/or alternative embodiment, the glucosamine-1-phosphate N-acetyltransferase activity and the N-acetylglucosamine-1-phosphate uridyltransferase activity are provided by a bifunctional enzyme. An example of such bifunctional enzyme is E. coli GlmU. E. coli GlmU catalyzes the last two sequential reactions which convert glucosamine-1-phosphate to UDP-GlcNAc (FIG. 1: \textcircled{3}). The C-terminal domain catalyzes the transfer of acetyl group from acetyl coenzyme A to glucosamine-1-phosphate (GlcN-1-P) to produce N-acetylglucosamine-1-phosphate (GlcNAc-1-P), which is converted into UDP-GlcNAc by the transfer of uridine 5-monophosphate (from uridine 5-triphosphate), a reaction catalyzed by the N-terminal domain.

Such bifunctional enzyme is also known from Bacillus subtilis and Haemophilus influenzae.

In an additional and/or alternative embodiment, the Bacillus cell has been genetically engineered to possess a heterologous bifunctional glucosamine-1-phosphate N-acetyltransferase/N-acetylglucosamine-1-phosphate uridyltransferase. A suitable example is E. coli GlmU or a functional variant thereof (including the H. influenzae GlmU). Said E. coli GlmU is encoded by the E. coli glmU gene.

In an additional and/or alternative embodiment, the Bacillus cell has been genetically engineered to contain and express or overexpress a nucleotide sequence which encodes said bifunctional enzyme, preferably a nucleotide sequence that encodes the E. coli GlmU or a functional variant thereof.

Thus, in an additional and/or alternative embodiment, the genetically engineered Bacillus cell contains a nucleic acid molecule comprising and expressing a nucleotide sequence which encodes the bifunctional glucosamine-1-phosphate N-acetyltransferase/N-acetylglucosamine-1-phosphate uridyltransferase. Hence, the nucleotide sequence which encodes a bifunctional glucosamine-1-phosphate N-acetyltransferase/N-acetylglucosamine-1-phosphate uridyltransferase is operably linked to at least one nucleic acid expression control sequence effecting transcription and/or translation of said nucleotide sequence which encodes a bifunctional glucosamine-1-phosphate N-acetyltransferase/N-acetylglucosamine-1-phosphate uridyltransferase in the genetically engineered Bacillus cell to provide intracellular glucosamine-1-phosphate N-acetyltransferase activity and N-acetylglucosamine-1-phosphate uridyltransferase activity.

In an additional and/or alternative embodiment, the codon usage of the nucleotide sequence encoding the heterologous bifunctional glucosamine-1-phosphate N-acetyltransferase/N-acetylglucosamine-1-phosphate uridyltransferase is adapted to the codon usage of Bacillus.

The recombinant bifunctional glucosamine-1-phosphate N-acetyltransferase/N-acetylglucosamine-1-phosphate uridyltransferase gene may be integrated into the Bacillus chromosome, or present as an episomal version on a plasmid within the Bacillus cell.

In an additional and/or alternative embodiment, the sialic acid biosynthesis pathway comprises the conversion of UDP-GlcNAc to N-acetylmannosamine (ManNAc). This conversion can be catalyzed by an UDP-N-acetylglucosamine 2-epimerase which does not just convert UDP-N-acetylglucosamine to UDP-N-acetylmannosamine, but releases UDP concomitantly (FIG. 1: \textcircled{4}).

Hence, the Bacillus cell comprises an UDP-N-acetylglucosamine 2-epimerase epimerase which releases UDP concomitantly.

A suitable UDP-N-acetylglucosamine 2-epimerase epimerase with concomitant release of UDP is the Campylobacter jejuni UDP-N-acetylglucosamine 2-epimerase NeuC, encoded by the C. jejuni neuC gene.

In an additional and/or alternative embodiment, the Bacillus cell has been genetically engineered to possess an UDP-N-acetylglucosamine 2-epimerase with concomitant release of UDP.

In an additional and/or alternative embodiment, the Bacillus cell has been genetically engineered to contain and express a nucleotide sequence which encodes a UDP-N-acetylglucosamine 2-epimerase which releases UDP concomitantly, preferably to contain and express a nucleotide sequence that encodes the C. jejuni NeuC or a functional variant thereof.

The C. jejuni UDP-N-acetylglucosamine 2-epimerase NeuC is encoded by the protein coding region of the C. jejuni neuC gene. Hence, the Bacillus cell has been genetically engineered to contain and express a nucleotide sequence that encodes the C. jejuni NeuC.

Thus, in an additional and/or alternative embodiment, the genetically engineered Bacillus cell contains a nucleic acid molecule comprising and expressing a nucleotide sequence which encodes the UDP-N-acetylglucosamine 2-epimerase with concomitant release of UDP during its enzymatic reaction to release ManNAc. Hence, the nucleotide sequence which encodes a UDP-N-acetylglucosamine 2-epimerase which releases UDP concomitantly is operably linked to at least one nucleic acid expression control sequence effecting transcription and/or translation of said nucleotide sequence which encodes said UDP-N-acetylglucosamine 2-epimerase in the genetically engineered Bacillus cell to provide intracellular UDP-N-acetylglucosamine 2-epimerase activity.

In an additional and/or alternative embodiment, the codon usage of the nucleotide sequence encoding the heterologous UDP-N-acetylglucosamine 2-epimerase with concomitant release of UDP is adapted to the codon usage of Bacillus.

The recombinant UDP-N-acetylglucosamine 2-epimerase gene may be integrated into the Bacillus chromosome, or present as an episomal version on a plasmid within the Bacillus cell.

In an alternative embodiment, the sialic acid biosynthesis pathway utilizes N-acetylglucosamine-6-phosphate (GlcNAc-6-P) as an intermediate, but not utilize UDP-GlcNAc. The genetically engineered Bacillus cell comprises a sialic acid biosynthesis pathway for the intracellular biosynthesis of N-acetylneuraminic acid which uses GlcNAc-6-P as intermediate comprises a glucosamine-6-phosphate N-acetyltransferase (FIG. 1: \textcircled{5}). A sialic acid biosynthesis pathway using a glucosamine-6-phosphate N-acetyltransferase for the intracellular biosynthesis of N-acetylneuraminic acid does not utilize UDP-GlcNAc for the biosynthesis of sialic acid.

The sialic acid biosynthesis pathway using GlcNAc-6P comprises the enzymatic activities of a glutamine:fructose-6-phosphate aminotransferase (FIG. 1: \textcircled{1}) and an N-acetylneuraminic acid synthase (FIG. 1: \textcircled{10}). Said sialic acid biosynthesis pathway further comprises a) the enzymatic activities of a glucosamine-6-phosphate N-acetyltransferase (FIG. 1: \textcircled{5}), an N-acetylglucosamine-6-phosphate phosphatase (FIG. 1: \textcircled{6}) and an N-acetylglucosamine 2-epimerase (FIG. 1: \textcircled{8}) or b) the enzymatic activities of a glucosamine-6-phosphate N-acetyltransferase (FIG. 1: \textcircled{5}), an N-acetylglucosamine-6-phosphate epimerase (FIG. 1: \textcircled{7}) and an N-acetylmannosamine-6-phosphate phosphatase (FIG. 1: \textcircled{9}). Therefore, it is not necessary that the genetically engineered Bacillus cell comprises the enzymatic activities of a phosphoglucosamine mutase, an N-acetylglucosamine-1-phosphate uridyltransferase and an UDP N-acetylglucosamine 2-epimerase with concomitant release of UDP for intracellular sialic acid biosynthesis. Thus, in an additional and/or alternative embodiment, the genetically engineered Bacillus cell being capable of synthesizing sialic acid does not comprise one or more enzymatic activities selected from the group consisting of the enzymatic activities of a phosphoglucosamine mutase, an N-acetylglucosamine-1-phosphate uridyltransferase and an UDP N-acetylglucosamine 2-epimerase with concomitant release of UDP.

In an additional and/or alternative embodiment, the genetically engineered Bacillus cell possesses glucosamine-6-phosphate N-acetyltransferase activity. Said glucosamine-6-phosphate N-acetyltransferase activity converts GlcN-6P to N-acetylglucosamine-6-phosphate (GlcNAc-6P). An example of a glucosamine-6-phosphate N-acetyltransferase is the Saccharomyces cerevisiae Gna1 (UniProtKB—P43577).

In an additional and/or alternative embodiment, the genetically engineered Bacillus cell contains a glucosamine-6-phosphate N-acetyltransferase, preferably a heterologous glucosamine-6-phosphate N-acetyltransferase, more preferably Gna1 from S. cerevisiae or a functional variant thereof.

Thus, in an additional and/or alternative embodiment, the genetically engineered Bacillus cell contains a nucleic acid molecule comprising and expressing a nucleotide sequence encoding a glucosamine-6-phosphate N-acetyltransferase, preferably S. cerevisiae Gna1, wherein said nucleotide sequence is operably linked to at least one nucleic acid expression control sequence effecting transcription and/or translation of said nucleotide sequence in the genetically engineered Bacillus cell to provide intracellular glucosamine-6-phosphate N-acetyltransferase activity.

In an additional and/or alternative embodiment, the codon usage of the nucleotide sequence encoding the heterologous glucosamine-6-phosphate N-acetyltransferase is adapted to the codon usage of Bacillus.

The recombinant glucosamine-6-phosphate N-acetyltransferase gene may be integrated into the Bacillus chromosome, or present as an episomal version on a plasmid within the Bacillus cell.

In an additional and/or alternative embodiment, the genetically engineered Bacillus cell possesses a N-acetylglucosamine-6-phosphate phosphatase activity. Said N-acetylglucosamine-6-phosphate phosphatase activity converts GlcNAc-6P to N-acetylglucosamine (GlcNAc). Examples of an N-acetylglucosamine-6-phosphate phosphatase are sugar phosphatases of the HAD-like superfamily which catalyze the conversion of GlcNAc6P to GlcNAc. The HAD-like superfamily of enzymes is named after the bacterial enzyme haloacid dehydrogenase and includes phosphatases. A suitable phosphatase of the HAD-like superfamily catalyzing the conversion of GlcNAc6P to GlcNAc may be selected from the group consisting of fructose-1-phosphate phosphatase (YqaB, UniProtKB—P77475) and alpha-D-glucose 1-phosphate phosphatase (YihX, UniProtKB—P0A8Y3). The E. coli YqaB and E. coli YihX enzymes are considered to also act on GlcNAc6P (Lee, S.-W. and Oh, M.-K. (2015) Metabolic Engineering 28: 143-150).

In an additional and/or alternative embodiment, the sugar phosphatase of the HAD-like superfamily catalyzing the conversion of GlcNAc-6P to GlcNAc is a heterologous enzyme in the genetically engineered Bacillus cell. In an additional and/or alternative embodiment, the sugar phosphatase of the HAD-like superfamily catalyzing the conversion of GlcNAc6P to GlcNAc is selected from the group consisting of E. coli YqaB, E. coli YihX, and functional variants thereof.

In an additional and/or alternative embodiment, the genetically engineered Bacillus cell contains a nucleic acid molecule which comprises and expresses a nucleotide sequence encoding a sugar phosphatase of the HAD-like superfamily catalyzing the conversion of GlcNAc6P to GlcNAc. In an additional and/or alternative embodiment, the nucleotide sequence encoding the sugar phosphatase of the HAD-like superfamily catalyzing the conversion of GlcNAc6P to GlcNAc is a heterologous nucleotide sequence. In an additional and/or alternative embodiment, the nucleotide sequence encoding the sugar phosphatase of the HAD-like superfamily catalyzing the conversion of GlcNAc6P to GlcNAc encodes the E. coli YqaB or E. coli YihX or a functional variant of one of these two enzymes.

In an additional and/or alternative embodiment, the codon usage of the nucleotide sequence encoding the heterologous N-acetylglucosamine-6-phosphate phosphatase is adapted to the codon usage of Bacillus.

The recombinant N-acetylglucosamine-6-phosphate phosphatase gene may be integrated into the Bacillus chromosome, or present as an episomal version on a plasmid within the Bacillus cell.

In an additional and/or alternative embodiment, the genetically engineered Bacillus cell possesses N-acetylglucosamine 2-epimerase activity. N-acetylglucosamine 2-epimerase (EC 5.1.3.8) is an enzyme that catalyzes the conversion of N-acetylglucosamine (GlcNAc) to N-acetylmannosamine (ManNAc). The enzyme is a racemase acting on carbohydrates and their derivatives. The systematic name of this enzyme class is N-acyl-D-glucosamine 2-epimerase. This enzyme participates in amino-sugar metabolism and nucleotide-sugar metabolism, preferably a heterologous N-acetylglucosamine 2-epimerase.

In an additional and/or alternative embodiment, the genetically engineered Bacillus cell comprises an N-acetylglucosamine 2-epimerase, preferably a heterologous N-acetylglucosamine 2-epimerase. Examples of N-acetylglucosamine 2-epimerases were described from Anabena variabilis, Acaryochloris sp., Nostoc sp., Nostoc punctiforme, Bacteroides ovatus or Synechocystis sp. An example of a suitable N-acetylglucosamine 2-epimerase is the N-acetylglucosamine 2-epimerase of B. ovatus ATCC 8483 (UniProtKB—A7LVG6) as encoded by gene BACOVA_01816. Another example is the N-acetylglucosamine 2-epimerase of Synechocystis sp. (strain PCC 6803) (UniProtKB—P74124) which is also known as renin-binding protein and is encoded by the s/r1975 gene.

In an additional and/or alternative embodiment, the genetically engineered Bacillus cell contains a nucleic acid molecule comprising a nucleotide sequence which encodes an N-acetylglucosamine 2-epimerase, preferably the N-acetylglucosamine 2-epimerase of B. ovatus ATCC 8483 or Synechocystis sp. (strain PCC 6803) or a functional variant thereof.

Hence, the nucleotide sequence which encodes the N-acetylglucosamine 2-epimerase is operably linked to at least one nucleic acid expression control sequence effecting transcription and/or translation of said nucleotide sequence which encodes a N-acetylglucosamine 2-epimerase in the genetically engineered Bacillus cell to provide intracellular N-acetylglucosamine 2-epimerase activity.

In an additional and/or alternative embodiment, the codon usage of the nucleotide sequence encoding the heterologous N-acetylglucosamine 2-epimerase is adapted to the codon usage of Bacillus.

The recombinant N-acetylglucosamine 2-epimerase gene may be integrated into the Bacillus chromosome, or present as an episomal version on a plasmid within the Bacillus cell.

In an additional and/or alternative embodiment, the genetically engineered Bacillus cell possesses N-acetylglucosamine-6-phosphate epimerase activity and N-acetylmannosamine-6-phosphate phosphatase activity. N-acetylglucosamine-6-phosphatase epimerase converts N-acetylglucosamine-6-phosphate (GlcNAc-6P) to N-acetylmannosamine-6-phosphate (ManNAc-6P), whereas N-acetylmannosamine-6-phosphate phosphatase dephosphorylates ManNAc-6P to give N-acetylmannosamine (ManNAc). Possessing N-acetylglucosamine-6-phosphate epimerase activity and N-acetylmannosamine-6-phosphate phosphatase activity provides an additional or alternative way for providing ManNAc for Neu5Ac production.

In an additional and/or alternative embodiment, the genetically engineered Bacillus cell contains an N-acetylglucosamine-6-phosphate epimerase. An example of a suitable N-acetylglucosamine-6-phosphate epimerase is E. coli NanE (UniprotKB P0A761) as encoded by the E. coli nanE gene.

Thus, in an additional and/or alternative embodiment, the genetically engineered Bacillus cell contains a nucleic acid molecule comprising and expressing a nucleotide sequence encoding an N-acetylglucosamine-6-phosphate epimerase, preferably a nucleotide sequence encoding E. coli NanE or a functional variant thereof.

Thus, in an additional and/or alternative embodiment, the genetically engineered Bacillus cell contains a nucleic acid molecule comprising and expressing a nucleotide sequence encoding the N-acetylglucosamine-6-phosphate epimerase, wherein said nucleotide sequence is operably linked to at least one nucleic acid expression control sequence effecting transcription and/or translation of said nucleotide sequence in the genetically engineered microbial cell to provide intracellular N-acetylglucosamine-6-phosphate epimerase activity.

In an additional and/or alternative embodiment, the codon usage of the nucleotide sequence encoding the heterologous N-acetylglucosamine-6-phosphate epimerase is adapted to the codon usage of Bacillus.

In an additional and/or alternative embodiment, the genetically engineered Bacillus cell contains an N-acetylmannosamine-6-phosphate phosphatase which converts ManNAc-6P to ManNAc.

Thus, in additional and/or alternative embodiment, the genetically engineered Bacillus cell contains a nucleic acid molecule comprising and expressing a nucleotide sequence encoding an N-acetylmannosamine-6-phosphate phosphatase. Hence, the nucleotide sequence which encodes the N-acetylmannosamine-6-phosphate phosphatase is operably linked to at least one nucleic acid expression control sequence effecting transcription and/or translation of said nucleotide sequence which encodes a N-acetylmannosamine-6-phosphate phosphatase in the genetically engineered Bacillus cell to provide intracellular N-acetylmannosamine-6-phosphate phosphatase activity.

In an additional and/or alternative embodiment, the codon usage of the nucleotide sequence encoding the heterologous N-acetylmannosamine-6-phosphate phosphatase is adapted to the codon usage of Bacillus.

The recombinant N-acetylmannosamine-6-phosphate phosphatase gene may be integrated into the Bacillus chromosome, or present as an episomal version on a plasmid within the Bacillus cell.

The genetically engineered Bacillus cell possesses cytidine 5′-monophospho-(CMP)-N-acetylneuraminic acid synthetase (FIG. 1: \textcircled{11}) activity for transferring cytidine 5′-monophosphate onto N-acetylneuraminic acid to generate a CMP-activated N-acetylneuraminic acid (CMP-NeuNAc). Several 5′-monophospho(CMP)-sialic acid synthetases are known in the art and have been described, e.g. 5′-monophospho-(CMP)-sialic acid synthetases from E. coli, Neisseria meningitidis, Campylobacter jejuni, Streptococcus sp., etc.

In an additional and/or alternative embodiment, the genetically engineered Bacillus cell contains a cytidine 5′-monophospho-(CMP)-N-acetylneuraminic acid synthetase, preferably a heterologous cytidine 5′-monophospho-(CMP)-N-acetylneuraminic acid synthetase, more preferably the N-acetylneuraminate cytidyltransferase NeuA from C. jejuni. The C. jejuni NeuA is encoded by the C. jejuni neuA gene.

Thus, in an additional and/or alternative embodiment, the genetically engineered Bacillus cell contains a nucleic acid molecule comprising and expressing a nucleotide sequence encoding a cytidine 5′-monophospho-(CMP)-N-acetylneuraminic acid synthetase, wherein said nucleotide sequence is operably linked to at least one nucleic acid expression control sequence effecting transcription and/or translation of said nucleotide sequence in the genetically engineered microbial cell to provide N-acetylneuraminate cytidyltransferase activity.

In an additional and/or alternative embodiment, the codon usage of the nucleotide sequence encoding the heterologous cytidine 5′-monophospho-(CMP)-N-acetylneuraminic acid synthetase is adapted to the codon usage of Bacillus.

The recombinant cytidine 5′-monophospho-(CMP)-N-acetylneuraminic acid synthetase gene may be integrated into the Bacillus chromosome, or present as an episomal version on a plasmid within the Bacillus cell.

The genetically engineered Bacillus cell possesses a sialyltransferase, preferably a heterologous sialyltransferase, and more preferably a sialyltransferase activity selected from the group consisting of α-2,3-sialyltransferase activity, a-2,6-sialyltransferase activity and/or α-2,8-sialyltransferase activity. The sialyltransferase activity is capable of transferring the N-acetylneuraminic acid moiety from the CMP-NeuNAc to an acceptor molecule, wherein said acceptor molecule is a saccharide molecule, to provide a sialylated saccharide.

In an additional and/or alternative embodiment, the genetically engineered microbial cell contains at least one sialyltransferase, preferably at least one heterologous sialyltransferase, wherein said sialyltransferase is capable of possessing an α-2,3-sialyltransferase activity and/or an a-2,6-sialyltransferase activity and/or an α-2,8-sialyltransferase activity for transferring the NeuNAc moiety from CMP-NeuNAc as donor substrate to the acceptor saccharide. Exemplary sialyltransferases and their genes are identified in Table 2.

The term “sialyltransferase” as used herein refers to polypeptides being capable of possessing sialyltransferase activity. “Sialyltransferase activity” refers to the transfer of a sialic acid residue, preferably of an N-acetylneuraminic acid (Neu5Ac) residue, from a donor substrate to an acceptor molecule. The term “sialyltransferase” comprises functional fragments of the sialyltransferases described herein, functional variants of the sialyltransferases described herein, and functional fragments of the functional variants. “Functional” in this regard means that the fragments and/or variants are capable of possessing sialyltransferase activity. Functional fragments of a sialyltransferase encompass truncated versions of a sialyltransferase as encoded by it naturally occurring gene, which truncated version is capable of possessing sialyltransferase activity. Examples of truncated versions are sialyltransferases which do not comprise a so-called leader sequence which typically directs the polypeptide to a specific subcellular localization. Typically, such leader sequences are removed from the polypeptide during its subcellular transport, and are also absent in the naturally occurring mature sialyltransferase.

The heterologous sialyltransferase is capable of transferring a sialic acid residue from a donor substrate to an acceptor molecule. The term “capable of” with respect to the heterologous sialyltransferase refers to the sialyltransferase activity of the heterologous sialyltransferase and the provision that suitable reaction conditions are required for the heterologous sialyltransferase to possess its enzymatic activity. In the absence of suitable reaction conditions, the heterologous sialyltransferase does not possess its enzymatic activity, but retains its enzymatic activity and possesses its enzymatic activity when suitable reaction conditions are restored. Suitable reaction conditions include the presence of a suitable donor substrate, the presence of suitable acceptor molecules, the presence of essential cofactors such as—for example—monovalent or divalent ions, a pH value in an appropriate range, a suitable temperature and the like. It is not necessary that the optimum values for each and every factor effecting the enzymatic reaction of the heterologous sialyltransferase is met, but the reaction conditions have to be such that the heterologous sialyltransferase performs its enzymatic activity. Accordingly, the term “capable of” excludes any conditions upon which the enzymatic activity of the heterologous sialyltransferase has been irreversibly impaired and also excluded exposure of the heterologous sialyltransferase to any such condition. Instead, “capable of” means that the sialyltransferase is enzymatically active, i.e. possesses its sialyltransferase activity, if permissive reactions conditions (where all requirements being necessary for the sialyltransferase to perform its enzymatic activity) are provided to the sialyltransferase.

Sialyltransferases can be distinguished on the type of sugar linkage they form. As used herein, the terms “α-2,3-sialyltransferase” and “α-2,3-sialyltransferase activity” refer to polypeptides and their enzymatic activity which add a sialic acid residue with an α-2,3 linkage to galactose, N-acetylgalactosamine or a galactose or N-acetylgalactosamine residue of the acceptor molecule. Likewise, the terms “α-2,6-sialyltransferase” and “α-2,6-sialyltransferase activity” refer to polypeptides and their enzymatic activity which add a sialic acid residue with an α-2,6 linkage to galactose, N-acetylgalactosamine or a galactose or N-acetylgalactosamine residue of the acceptor molecule. Likewise, the terms “α-2,8-sialyltransferase” and “α-2,8-sialyltransferase activity” refer to polypeptides and their enzymatic activity which add a sialic acid residue with an α-2,8 linkage to galactose, N-acetylgalactosamine or a galactose or N-acetylgalactosamine residue of the acceptor molecule.

In addition, the genetically engineered Bacillus cell has been genetically engineered to contain and express the nucleotide sequence encoding the heterologous sialyltransferase. To this end, the nucleotide sequence encoding the heterologous sialyltransferase is operably linked to at least one expression control sequence effecting transcription and/or translation of said nucleotide sequence encoding the heterologous sialyltransferase in the genetically engineered Bacillus cell to provide intracellular sialyltransferase activity.

In another embodiment, the heterologous sialyltransferase is capable of possessing α-2,6-sialyltransferase activity.

In an additional and/or alternative embodiment, the genetically engineered Bacillus cell contains and expresses a nucleic acid molecule which comprises at least one nucleotide sequence encoding said heterologous sialyltransferase being capable of possessing α-2,6-sialyltransferase activity.

In an additional and/or alternative embodiment, the heterologous sialyltransferase is capable of possessing α-2,8-sialyltransferase activity. An example of a heterologous sialyltransferase is capable of possessing α-2,8-sialyltransferase activity is the sialyltransferase CstII of Campylobacter jejuni OH4384.

The sialyltransferase is capable of transferring a sialic acid residue, e.g. a N-acetylneuraminic acid (Neu5Ac) residue, from a donor substrate, e.g. CMP-Neu5Ac, to an acceptor molecule. The acceptor molecule is a saccharide molecule, preferably a saccharide molecule set forth in Table 3.

In an additional and/or alternative embodiment, the genetically engineered microbial cell contains a nucleic acid molecule which comprises and expresses at least one nucleotide sequence encoding said heterologous sialyltransferase being capable of possessing α-2,3-sialyltransferase activity.

| | | Cloned as full |
|---|---|---|
| | accession | length (FL) |
| | number of the | gene or |
| | sialyltransferase | without signal |
| Origin of the sialyltransferase gene | gene | peptide (Δ) |
| Neisseria meningitidis | U60660 | FL |
| Campylobacter jejuni strain OH4384 | AF130466 | FL |
| Campylobacter jejuni strain OH4384 | AX934425 | FL |
| Helicobacter acinonychis | NC_008229 | FL |
| Helicobacter acinonychis | NC_008229 | FL |
| Photobacterium sp. JT-ISH-224 | BAF92026 | Δ17 |
| Pasteurella dagmatis strain DSM 22969 | AFY98851 | FL |
| Photobacterium sp. JT-ISH-224 | BAF92025 | Δ20 |
| Vibrio sp. JT-FAJ-16 | BAF91160 | Δ22 |
| Pasteurella multocida PM70 | AAK02272 | Δ25 |
| Photobacterium damselae JT0160 | BAA25316 | FL |
| Streptococcus agalactiae | AB050723 | FL |
| Haemophilus-somnus-2336 | ACA31578 | FL |
| Haemophilus ducreyi 35000HP | AF101047 | FL |
| Haemophilus ducreyi 35000HP | AAP95068 | FL |
| Photobacterium phosphoreum JT-ISH-467 | BAF63530 | Δ20 |
| Photobacterium leiognathi JT-SHIZ-119 | AB500947 | Δ15 |
| Photobacterium leiognathi JT-SHIZ-145 | BAF91416 | Δ15 |
| Campylobacter coli | YP_008473374 | FL |
| Vibrio harveyi | WP_017817635 | Δ24 |
| Streptococcus entericus | WP_018369230 | FL |
| Avibacterium paragallinarum | WP_021724759 | FL |
| Haemophilus parahaemolyticus HK385 | EIJ71207 | FL |
| Alistipes sp. CAG:268 | CDC95697 | Δ17 |
| Alistipes sp. AL-1 | WP_032134786 | FL |
| Pasteurella multocida PM70 | NC_002663 | FL |
| Campylobacter jejuni strain 81-176 | AAL09368 | FL |
| Alistipes shahii WAL 8301 | YP_007816735 | Δ21 |
| Actinobacillus suis ATCC 33415 | AIJ32009 | FL |
| Actinobacillus capsulatus DSM 19761 | WP_018652686 | FL |
| Bibersteinia trehalosi USDA-ARS-USMARC-189 | AHG84654 | FL |
| Photobacterium damselae subsp. damselae CIP | EEZ40509 | FL |
| 102761 | | |
| Haemophilus somnus 2336 | ACA31170 | FL |
| Streptococcus suis | AGL48117 | FL |
| Streptococcus suis | EEF64118 | FL |
| Photobacterium phosphoreum | CDN97322 | |
| Alistipes timonensis | WP_026020701 | |
| Haaemophilus parasuis | KEZ22922 | |
| Acinetobacter pittii | EOQ74854 | |
| Photobacterium leiognathi | BAI49484 | |
| Porphyroonas sp. | KGO0080 | |
| Photobacterium damsalae | WP_005298232 | |
| Pasteurella multocida | EGP04633 | |
| Actinobacillus ureae | WP_005624339 | |
| Actinobacillus equuli | WP_039196193 | |
| Acinetobacter calcoaceticus | WP_017391462 | |
| Acinetobacter baumannii | WP_032039596 | |
| Acinetobacter oleivorans | WP_042898369 | |
| Acinetobacter baumannii | ELX04805 | |
| Acinetobacter gyllenbergii | WP_032865812 | |
| Shigella boydii | ACD37071 | |
| Kingella kingae | WP_026036248 | |
| Alysiella crassa | WP_034294366 | |
| Alistipes sp. | EFR56292 | |
| Streptococcus suis | WP_024397787 | |
| Fusobacterium mortiferum | EEO36694 | |
| Porphyromonas catoniae | WP_005467220 | |

Table 2: List of sialyltransferase-encoding nucleotide sequences. The sialyltransferase-encoding nucleotide sequences were either cloned as full length constructs (FL) or without a predicted signal peptide (A) as compared to their wild-type protein coding regions. The number behind the Δ indicates the N-terminal amino acids deleted from the corresponding sequence.

In an additional and/or alternative embodiment, the acceptor molecule is a monosaccharide, preferably a monosaccharide selected from the group consisting of N-acetylglucosamine, galactose and N-acetylgalactosamine.

In an additional and/or alternative embodiment, the acceptor molecule is a disaccharide, preferably a disaccharide selected from the group consisting lactose, lactulose, N-acetyllactosamine, lacto-N-biose, lactulose and melibiose.

In an additional and/or alternative embodiment, the acceptor molecule is a trisaccharide, preferably a trisaccharide selected from the group consisting of raffinose, lacto-N-triose II, 2′-fucosyllactose, 3-fucosyllactose, 3′-sialyllactose, 6′-sialyllactose, 3′-sialyl-N-acetyllactosamine, 6′-sialyl-N-acetyllactosamine, 3′-galactosyllactose and 6′-galactosyllactose.

| Name | Abbreviation | Structure |
|---|---|---|
| N-acetyl- | GlcNAc | GlcNAc |
| glucosamine | | |
| Galactose | Gal | Gal |
| N- | GalNAc | GalNAc |
| acetylgalactosamine | | |
| Lactose | Lac | Gal(β1,4)Glc |
| N-acetyllactosamine | LacNAc | Gal(β1,4)GlcNAc |
| Lacto-N-biose | LNB | Gal(β1,3)GlcNAc |
| Lactulose | LacU | Gal(β1,4)Frc |
| Melibiose | Mel | Gal(α1,6)Glc |
| Raffinose | Raf | Gal(α1,6)Glc(α1,2)Frc |
| 2′-Fucosyllactose | 2′-FL | Fuc(α1,2)Gal(β1,4)Glc |
| 3-Fucosyllactose | 3-FL | Gal(β1,4)[Fuc(α1,3)]Glc |
| 2′,3- | DFL | Fuc(α1,2)Gal(β1,4)[Fuc(α1,3)]Glc |
| Difucosyllactose | | |
| 6′-Galactosyllactose | 6′-GL | Gal(β1,6)Gal(β1,4)Glc |
| 3′-Galactosyllactose | 3′-GL | Gal(β1,3)Gal(β1,4)Glc |
| Lacto-N-triose II | LNT II | GlcNAc(β1,3)Gal(β1,4)Glc |
| Lacto-N-tetraose | LNT | Gal(β1,3)GlcNAc(β1,3)Gal(β1,4)Glc |
| Lacto-N- | LNnT | Gal(β1,4)GIcNAc(β1,3)Gal(β1,4)Glc |
| neotetraose | | |
| Lacto-N- | LNFP I | Fuc(α1,2)Gal(β1,3)GlcNAc(β1,3)Gal(β1,4)Glc |
| fucopentaose I | | |
| Lacto-N- | LNnFP I | Fuc(α1,2)Gal(β1,4)GlcNAc(β1,3)Gal(β1,4)Glc |
| neofucopentaose I | | |
| Lacto-N- | LNFP II | Gal(β1,3)[Fuc(α1 ,4)]GlcNAc(β1,3)Gal(β1,4)Glc |
| fucopentaose II | | |
| Lacto-N- | LNFP III | Gal(β1,4)[Fuc(α1 ,3)]GlcNAc(β1,3)Gal(β1,4)Glc |
| fucopentaose III | | |
| Lacto-N- | LNFP V | Gal(β1,3)GlcNAc(β1,3)Gal(β1,4)[Fuc(α1,3)]Glc |
| fucopentaose V | | |
| Lacto-N- | LNnFP V | Gal(β1,4)GlcNAc(β1,3)Gal(β1,4)[Fuc(α1,3)]Glc |
| neofucopentaose | | |
| V | | |
| Lacto-N- | LNDH I | Fuc(α1,2)Gal(β1,3)[Fuc(α1,4)]GlcNAc(β1,3)Gal(β1,4)Glc |
| difucohexaose I | | |
| Lacto-N- | LND | Gal(β1,3)[Fuc(α1,4)]GlcNAc(β1,3)Gal(β1,4)[Fuc(α1,3)]Glc |
| difucohexaose II | | |
| Lacto-N- | LNnDFH 1 | Gal(β1,4)[Fuc(α1,3)]GlcNAc(β1,3)Gal(β1,4)[Fuc(α1,3)]Glc |
| neodifucohexaose | | |
| I | | |
| Lacto-N-hexaose | LNH | Gal(β1,4)GlcNAc(β1,6)[Gal(β1,3)GlcNAc(β1,3)]Gal(β1,4)Glc |
| Lacto-N- | LNnH | Gal(β1,4)GlcNAc(β1,6)[Gal(β1,4)GlcNAc(β1,3)]Gal(β1,4)Glc |
| neohexaose | | |
| para-Lado-N- | paraLNT | Gal(β1,3)GlcNAc(β1,3)Gal(β1,4)GlcNAc(β1,3)Gal(β1,4)Glc |
| hexaose | | |
| para-Lado-N- | paraLNnH | Gal(β1,4)GlcNAc(β1,3)Gal(β1,4)GlcNAc(β1,3)Gal(β1,4)Glc |
| neohexaose | | |
| 3′-Sialyllactose | 3′-SL | Neu5Ac(α2,3)Gal(β1,4)Glc |
| 6′-Sialyllactose | 6′-SL | Neu5Ac(α2,6)Gal(β1,4)Glc |
| Sialyllacto-N- | LSTa | Neu5Ac(α2,3)Gal(β1,3)GlcNAc(β1,3)Gal(β1,4)Glc |
| tetraose a | | |
| Sialyllacto-N- | LSTb | Gal(β1,3)[Neu5Ac(α2,6)]GlcNAc(β1,3)Gal(β1,4)Glc |
| tetraose b | | |
| Sialyllacto-N- | LSTc | Neu5Ac(α2,6)Gal(β1,4)GlcNAc(β1,3)Gal(β1,4)Glc |
| tetraose c | | |
| Fucosyllacto-N- | F-LST-a | Neu5Ac(α2,3)Gal(β1,3)[Fuc(α1 ,4)]GlcNAc(β1,3)Gal(β1,4)Glc |
| sialylpentaose a | | |
| Fucosyllacto-N- | F-LST-b | Fuc(α1,2)Gal(β1,3)[Neu5Ac(α2,6)]GlcNAc(β1,3)Gal(β1,4)Glc |
| sialylpentaose b | | |
| Fucosyllacto-N- | F-LST-c | Neu5Ac(α2,3)Gal(β1,3)GlcNAc(β1,3)Gal(β1,4)[Fuc(α1,3)]Glc |
| sialylpentaose c | | |
| Disialyllacto-N- | DS-LNT | Neu5Ac(α2,3)Gal(β1,3)[Neu5Ac(α2,6)]GlcNAc(β1,3)Gal(β1,4)Glc |
| tetraose | | |
| 3-Fucosyl-3′- | 3F-3-SL | Neu5Ac(α2,3)Gal(β1,4)[Fuc(α1,3)]Glc |
| sialyllactose | | |
| 3-Fucosyl-6′- | 3F-6-SL | Neu5Ac(α2,6)Gal(β1,4)[Fuc(α1,3)]Glc |
| sialyllactose | | |
| 3′-sialyl-N- | 3-SLN | Neu5Ac(α2,3)Gal(β1,4)GlcNAc |
| acetyllactosamine | | |
| 6′-sialyl-N- | 6-SLN | Neu5Ac(α2,6)Gal(β1,4)GlcNAc |
| acetyllactosamine | | |

Table 3: List of saccharides that may be used as acceptor substrate for the production of a sialylated saccharide. The sialylated oligosaccharide itself may also be used as acceptor substrate for the production of a further sialylated oligosaccharide.

In an additional and/or alternative embodiment, the acceptor molecule is a tetrasaccharide, preferably a tetrasaccharide selected from the group consisting of lacto-N-tetraose, lacto-N-neotetraose, 2′3-difucosyllactose, 3-fucosyl-3′-sialyllactose and 3-fucosyl-6′-sialyllactose.

In an additional and/or alternative embodiment, the acceptor molecule is a pentasaccharide, preferably a pentasaccharide selected from the group consisting of sialyllacto-N-tetraose a, sialyllacto-N-tetraose b, sialyllacto-N-tetraose c, lacto-N-fucopentaose I, lacto-N-fucopentaose II, lacto-N-fucopentaose III, lacto-N-fucopentaose V, lacto-N-neofucopentaose I and lacto-N-neofucopentaose V.

It is to be understood that a Bacillus cell already carrying one or more genes encoding said enzymes, and expressing said genes in a manner sufficient to produce NeuNAc, CMP-NeuNAc and/or the sialylated saccharide does not need to be genetically engineered to complete sialic acid biosynthesis and to transfer a sialic acid moiety to a saccharide acceptor, but may nevertheless be genetically engineered to alter the expression level of one or more of said genes to increase the intracellular level of said one or more gene products such as—for example the quantity of glutamine:fructose-6-phosphate aminotransferase, glucosamine-6-phosphate N-acetyltransferase, N-acetylglucosamine-6-phosphate phosphatase, N-acetylglucosamine 2-epimerase and/or N-acetylneuraminic acid synthase, thus increasing the rate of Neu5Ac biosynthesis and, as a consequence, of the sialylated saccharide, in the genetically engineered cell.

In some embodiments, the Bacillus cell is for use in the production of 3′-SL and has been genetically engineered to possess a lactose permease, a CMP-NeuNAc biosynthesis pathway that uses GlcN-1P as intermediate, and a α-2,3-sialyltransferase.

In various embodiments, the Bacillus cell is for use in the production of 6′-SL and has been genetically engineered to possess a lactose permease, a CMP-NeuNAc biosynthesis pathway that uses GlcN-1P as intermediate, and a α-2,6-sialyltransferase.

In an additional and/or alternative embodiment, the genetically engineered Bacillus cell synthesizes more PEP than the wildtype of the cell. In an additional and/or alternative embodiment, the genetically engineered microbial cell has been genetically engineered to possess an enhanced PEP biosynthesis pathway. Preferably, the genetically engineered microbial cell has been genetically engineered to possess an increased phosphoenolpyruvate synthase activity, for example in that the ppsA gene encoding phosphoenolpyruvate synthase gene is overexpressed and/or in that the non-naturally-occurring microorganisms contains at least one additional copy of a nucleotide sequence allowing the expression of a phosphoenolpyruvate synthase or a functional variant thereof. Overexpression of ppsA enhances intracellular PEP synthesis such that more PEP is available for the production of sialic acid. For example, a suitable phosphoenolpyruvate synthase is PpsA of E. coli.

In an additional and/or alternative embodiment, the genetically engineered Bacillus cell contains a nucleic acid molecule comprising a nucleotide sequence encoding E. coli PpsA or a functional variant thereof. Said nucleotide sequence encoding E. coli PpsA or a functional variant thereof has a sequence identity of at least 80%, at least 85%, at least 90%, at least 95%, at least 98% or at least 99% to the E. coli ppsA gene.

In an additional and/or alternative embodiment, the genetically engineered Bacillus cell is further modified to be capable to transfer said sole carbon source into the cell via a mechanism, that is not consuming PEP.

In an additional and/or alternative embodiment, the Bacillus cell for producing a sialylated oligosaccharide lacks any β-galactosidase activity or possesses reduced β-galactosidase activity as compared to a wild type Bacillus cell of the same species.

The intracellular biosynthesis of sialylated oligosaccharides requires import of lactose as acceptor substrate for the lactose-accepting sialyltransferase. Any intracellular enzyme activity that hydrolyzes internalized lactose would affect the efficacy of sialyllactose formation as the pool of intracellular lactose would be diminished. Therefore, it would be advantageous if the Bacillus cell for producing a sialylated oligosaccharide would lack or at least possess—as compared to the wild-type Bacillus cell—reduced beta-galactosidase activity.

In an additional and/or alternative embodiment, the Bacillus cell has been genetically engineered to abolish or at least reduce the cell's β-galactosidase activity.

In an additional and/or alternative embodiment, the Bacillus cell has been genetically engineered by deletion or functional inactivation of the ganA gene. In another embodiment, the Bacillus cell has been genetically engineered to reduce the expression level of the ganA gene—as compared to the wild-type Bacillus cell.

The Bacillus ganA gene is also called yvfN or lacA. It is a gene of the GanR regulon which contains genes encoding enzymes involved in the utilization of galactan. The ganA gene encodes a beta-galactosidase that is involved in the galactan utilization of Bacillus.

Deletion or functional inactivation of the ganA gene abolishes the GanA-mediated β-galactosidase activity in the Bacillus cell, whereas reduction of the ganA expression lowers the amount of GanA in the Bacillus cell and hence the β-galactosidase activity which could interfere with the biosynthesis of a sialylated oligosaccharide.

In an additional and/or alternative embodiment, the Bacillus cell has been genetically engineered by deletion or functional inactivation of the yesZ gene. The Bacillus yesZ gene encodes the beta-galactosidase YesZ which plays a role in the degradation of rhamnogalacturonan being derived from plant cell walls. The expression of Bacillus yesZ gene is induced by rhamnogalacturonan I. In another embodiment, the Bacillus cell has been genetically engineered to reduce the expression level of the yesZ gene—as compared to the wild-type Bacillus cell.

Deletion or functional inactivation of the yesZ gene abolishes the YesZ-mediated β-galactosidase activity in the Bacillus cell, whereas reduction of the yesZ expression lowers the amount of YesZ in the Bacillus cell and hence the β-galactosidase activity which could interfere with the biosynthesis of a sialylated oligosaccharide.

When B. subtilis enters the post-exponential growth phase, they (start to) produce large quantities of extracellular proteases. Foreign proteins are often protease sensitive. Therefore, an exoprotease-free strain is desirable to increase the stability of heterologous proteins and to allow accumulation of high levels of foreign proteins.

The genome of Bacillus encodes for at least eight extracellular proteases, namely nprE, aprE, epr, bpr, mpr, nprB, vpr and wprA. Thus, in an additional and/or alternative embodiment, the Bacillus cell has been genetically engineered in that at least one gene encoding for an extracellular protease has been deleted or functionally inactivated, preferably at least one of the genes selected from the group consisting of nprE, aprE, epr, bpr, mpr, nprB, vpr and wprA. Preferably, two, three, four, five six, seven or eight of the genes selected from the group consisting of nprE, aprE, epr, bpr, mpr, nprB, vpr and wprA have been deleted or functionally inactivated.

B. subtilis synthesizes pulcherriminic acid when growing in media containing a carbohydrate such as glucose or lactose. Excreted pulcherriminic acid forms the red pigment pulcherrimin, the salt of pulcherriminic acid (a ferric chelate), when iron is present in the growth medium. The formation of this undesired by-product during fermentation processes can be avoided/abolished by deletion or disruption of the genes yvmC and/or cypX. The gene yvmC encodes a cyclodipeptide synthase and the gene cypX encodes a cytochrome P450 cyclo-l-leucyl-1-leucyl dipeptide oxidase.

Thus, in an additional and/or alternative embodiment, the Bacillus cell has been genetically engineered in that at least one of the genes yvmC and cypX has been deleted or functionally inactivated.

The rhizobacterium B. subtilis possesses genes for the synthesis of more than 20 antibiotics. Among them are peptide antibiotics like Bacillus subtilis lantibiotics and lantibiotic-like peptides (subtilin, ericin S, mersacidin, sublancin 168, subtilosin A) and non-ribosomally synthesized (peptide) antibiotics (surfactin, iturin, bacillomycin, mycosubtilin, corynebactin/bacillibactin, fengycin plipastatin, mycobacillin, TL-119, bacilysin, bacilysocin, amicoumacin, 3,3′-neotrehalosadiamine, difficidin, rhizocticin).

For producing a sialylated oligosaccharide, it is preferred to use a Bacillus cell that does not produce an antibiotic. Thus, in an additional and/or alternative embodiment, the Bacillus cell does not synthesize one or more of the antibiotics selected from the group consisting of lantibiotics and lantibiotic-like peptides such as subtilin, ericin S, mersacidin, sublancin 168, subtilosin A; non-ribosomally synthesized (peptide) antibiotics such as surfactin, iturin, bacillomycin, mycosubtilin, corynebactin/bacillibactin, fengycin plipastatin, mycobacillin, TL-119, bacilysin, bacilysocin, amicoumacin, 3,3′-neotrehalosadiamine, difficidin and rhizocticin. The Bacillus cell may have been genetically engineered to impair or abolish their ability to synthesize one or more of said antibiotics.

The genetically engineered Bacillus cell that can produce sialylated oligosaccharides may—optionally—include additional features, and may be genetically engineered to possess these additional features. These additional features are considered to improve the productivity of the non-naturally-occurring microorganism leading to higher sialylated oligosaccharide yields.

The Bacillus cell of the invention is able to produce a sialylated oligosaccharide when cultivated in the presence of lactose in a medium and under conditions that are permissive for the Bacillus cell to produce the sialylated oligosaccharide. Thus, provided is also the use of a genetically engineered Bacillus cell as described herein before for the production of a sialylated oligosaccharide.

In an aspect, provided is a method for the production of a sialylated oligosaccharide. The method comprises (i) providing a Bacillus cell as describe herein before, (ii) cultivating said Bacillus cell in a culture medium or fermentation broth and under conditions that are permissive for the production of the sialylated oligosaccharide. The method may further include retrieving the sialylated oligosaccharide from the culture medium/fermentation broth and/or from the Bacillus cell.

The fermentation broth contains at least one carbon source for the Bacillus cell's metabolism. In some embodiments, the at least one carbon source is selected from the group consisting of glucose, fructose, sucrose, glycerol and combinations thereof. The carbon source can be internalized by the Bacillus cell and may be sued by the Bacillus cell's metabolism to generate biomass and/or energy in form of energy rich triphosphates.

In some embodiments, the fermentation broth contains lactose, in particular if the Bacillus cell is not able to synthesize lactose by its own. The lactose may be supplied exogenously to the fermentation broth. Lactose may serve as acceptor substrate for a NeuNAc moiety in the production of some sialylated oligosaccharides, in particular in the production of sialyllactoses.

In various embodiments, the fermentation broth contains sialic acid, in particular if a Bacillus cell is used for the production of a sialylated oligosaccharide which does not possess a sialic acid biosynthesis pathway, but a sialic acid salvage pathway.

In some embodiments, the production of a sialylated oligosaccharide does not require addition of N-acetylglucosamine, N-acetylmannosamine and/or N-acetylneuraminic acid to the fermentation broth and/or cultivating the genetically engineered microbial cell in the presence of N-acetylglucosamine, N-acetylmannosamine and/or N-acetylneuraminic acid for the intracellular biosynthesis of the sialylated oligosaccharide, because a Bacillus cell is used for the production of the sialylated oligosaccharide which possesses a de novo sialic acid biosynthesis pathway for intracellular biosynthesis of NeuNAC.

In the method, the at least one genetically engineered Bacillus cell is cultivated in a fermentation broth and under conditions which are permissive for the production of the saccharide comprising at least one N-acetylneuraminic acid moiety.

While the process employs a carbon source in the fermentation broth for the genetically engineered Bacillus cell, it is not necessary to add glucosamine and/or N-acetylneuraminic acid and/or N-acetylglucosamine and/or N-acetylmannosamine to the fermentation broth when the Bacillus cell is able to intracellularly produce N-acetylneuraminic acid. Thus, in various embodiments for the production of a sialylated oligosaccharide, the genetically engineered Bacillus cell is cultivated in the absence of and/or without addition of one or more selected from the group consisting of glucosamine, N-acetylglucosamine, N-acetylmannosamine and N-acetylneuraminic acid. The genetically engineered Bacillus cell may be cultivated in the absence and/or without addition of galactose, as far as galactose is not supplied as an acceptor substrate for the sialyltransferase reaction.

In additional and/or alternatives embodiments, the genetically engineered Bacillus cell is cultivated in the presence of one or more monosaccharides (e.g. galactose), disaccharides (e.g. lactose), trisaccharides (e.g. lacto-N-triose II), tetrasaccharides (e.g. lacto-N-tetraose) and/or pentasaccharides (e.g. sialyllacto-N-tetraose a).

According to an additional and/or alternative embodiment, the genetically engineered Bacillus cell is cultivated in the presence of at least one acceptor substrate selected from the group consisting of galactose, N-acetylgalactosamine, N-acetylglucosamine, lactose, lactulose, N-acetyllactosamine, lacto-N-biose, lacto-N-triose, 2′-fucosyllactose, 3-fucosyllactose, 3′-sialyllactose, 6′-sialyllactose, 3′-sialyl-N-acetyllactosamine, 6′-sialyl-N-acetyllactosamine, 3′-galactosyllactose, 6′-galactosyllactose, lacto-N-triose II, lacto-N-tetraose, lacto-N-neotetraose, 2′3-difucosyllactose, 3-fucosyl-3′-sialyllactose and 3-fucosyl-6′-sialyllactose. These substrates are imported into the cell and used as acceptor molecules in the cell.

The method comprises the optional step of recovering the sialylated oligosaccharide that has been produced by the genetically engineered Bacillus cell during its cultivation and proliferation in the fermentation broth. The sialylated oligosaccharide can be recovered from the fermentation broth after the genetically engineered Bacillus cells have been removed, for example by centrifugation or filtration, and/or can be recovered from the cells, for example in that the cells are harvested from the fermentation broth by centrifugation and are subjected to a cell lysis step. Subsequently, the sialylated oligosaccharides can be further purified from the fermentation broth and/or cell lysates by suitable techniques known to the skilled artisan. Suitable techniques include microfiltration, ultrafiltration, diafiltration, simulated moving bed type chromatography, electrodialysis, reverse osmosis, gel filtration, anion exchange chromatography, cation exchange chromatography, and the like.

The method and the genetically engineered microbial cell that is employed in the method are used for the production of a sialylated oligosaccharide. The term “sialylated oligosaccharide” refers to an oligosaccharide molecule comprising at least one N-acetylneuraminic acid moiety.

In an additional and/or alternative embodiment, the sialylated saccharide is an oligosaccharide. The term “oligosaccharide” as used herein refers to polymers of monosaccharide residues, wherein said polymers comprise at least two monosaccharide residues, but no more than 10 monosaccharide residues, preferably no more than 7 monosaccharide residues. The oligosaccharides are either a linear chain of monosaccharides or are branched. In addition, the monosaccharide residues of the oligosaccharides may feature a number of chemical modifications. Accordingly, the oligosaccharides may comprise one or more non-saccharide moieties. The term “sialylated oligosaccharide” as used herein refers to oligosaccharides comprising one or more N-acetylneuraminic acid moieties.

According to additional and/or alternative embodiment, the sialylated oligosaccharide is selected from the group consisting of 3′-sialyllactose, 6′-sialyllactose, sialyllacto-N-tetraose a, sialyllacto-N-tetraose b, sialyllacto-N-tetraose c, fucosyl-sialyllacto-N-tetraose a, fucosyl-sialyllacto-N-tetraose b, fucosyl-sialyllacto-N-tetraose c, disialyllacto-N-tetraose, fucosyldisialyllacto-N-tetraose I, fucosyldisialyllacto-N-tetraose II, 3′-sialylgalactose, 6′-sialylgalactose, 3′-sialyl-N-acetyllactosamine and 6′-sialyl-N-acetyllactosamine.

In another aspect of the invention, the use of a genetically engineered Bacillus cell as described herein before for the production of a sialylated oligosaccharide in a whole cell fermentation process is provided, i.e. the sialylated oligosaccharide is synthesized by and in the genetically engineered Bacillus cell.

In another aspect of the invention, a sialylated oligosaccharide is provided that has been produced by the method and/or by using the genetically engineered Bacillus cell as described herein before.

In another aspect of the invention, provided is the use of a sialylated oligosaccharide that has been produced by a method as described herein before and/or by using the genetically engineered Bacillus cell as described herein before for the manufacture of a nutritional composition.

Thus, provided is a nutritional composition containing at least one sialylated oligosaccharide which has been produced by the method and/or by the genetically engineered Bacillus cell as described herein before.

In an additional and/or alternative embodiment, the sialylated oligosaccharide is selected from the group consisting of 3′-sialyllactose, 6′-sialyllactose, sialyllacto-N-tetraose a, sialyllacto-N-tetraose b, sialyllacto-N-tetraose c, fucosyl-sialyllacto-N-tetraose a, fucosyl-sialyllacto-N-tetraose b, fucosyl-sialyllacto-N-tetraose c, disialyllacto-N-tetraose, fucosyldisialyllacto-N-tetraose I, fucosyldisialyllacto-N-tetraose II, 3′-sialylgalactose, 6′-sialylgalactose, 3′-sialyl-N-acetyllactosamine and 6′-sialyl-N-acetyllactosamine.

In some embodiments, the nutritional composition further contains at least one neutral HMO, preferably at least one neutral HMO that has been produced by using a genetically engineered Bacillus cell. The at least one neutral HMO is selected from the group consisting of 2′-fucosyllactose, 3-fucosyllactose, 2′,3-difucosyllactose, lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-neofucopentaose V, lacto-N-difucohexaose I, lacto-N-difucohexaose II, lacto-N-neodifucohexaose I, lacto-N-hexaose, lacto-N-neohexaose, para-lacto-N-hexaose, and para-lacto-N-neohexaose.

In an additional embodiment, the nutritional composition is selected from the group consisting of medicinal compositions, pharmaceutical compositions, neutraceutical formulations, infant formula and dietary supplements.

The nutritional composition may be present in liquid form or in solid form including, but not limited to, powders, granules, flakes and pellets.

The present invention will be described with respect to particular embodiments, but the invention is not limited thereto but only by the claims. Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description and drawings provided herein, numerous specific details are set forth. However, it is understood that embodiments of the invention may be practiced without these specific details. In other instances, well-known methods, structures and techniques have not been shown in detail in order not to obscure an understanding. The invention will now be described by a detailed description of several embodiments of the invention. It is clear that other embodiments of the invention can be configured according to the knowledge of persons skilled in the art without departing from the true spirit or technical teaching of the invention, the invention being limited only by the terms of the appended claims.

### EXAMPLES

### Example 1: Transformation of Bacillus subtilis

Bacillus subtilis can be genetically manipulated by various techniques. For transformation of B. subtilis, competent cells were prepared by a modified protocol of the two-step method (Anagnostopoulos, C. and Spizizen, J. (1961) J Bacteriol 81 (5): 741-746). An overnight culture was inoculated in MG1 medium and shaken at 37° C. MG1 medium is Spizizen's minimal medium, which is supplemented with 0.5% glucose, 5 mM MgSO₄ and 0.02% casamino acids (optionally additionally supplemented with biotin and/or L-tryptophan). At the next morning, this culture was diluted 1:20 in fresh MG1 medium and incubated at 37° C. for approximately 6 h. 1 ml of the culture was diluted in 8 ml MG2 medium which differs from MG1 medium in the concentration of casamino acids (0.01% instead of 0.02%). In a shortened protocol the overnight culture is directly diluted in MG2 medium. After incubation for another 90 min, a 1-ml portion of the culture was mixed with 1-3 μg multimeric plasmid DNA or linear DNA and incubated at 37° C. for 30-60 min with shaking. Multimeric plasmid DNA was obtained either by using E. coli strain NM538 for propagation of plasmid DNA or by linearization of the plasmid by digestion with a single-cutter restriction enzyme, which cleaves within the backbone, followed by re-ligation with T4 DNA ligase.

Afterwards, cells were spread on 2×YT agar plates containing the appropriate antibiotic. Antibiotics were added in the following concentrations: 5 μg·mL⁻¹ erythromycin, 5 μg·mL⁻¹ chloramphenicol, 10 μg·mL⁻¹ kanamycin, 100 μg·mL⁻¹ spectinomycin.

Alternatively, for protoplast transformation (Romero, D. et al. (2006) Journal of Microbiological Methods 66:556-559), cells were grown in 20 ml of Penassay broth (PAB) at 37° C. until the onset of the stationary phase of growth (OD₆₀₀=1.7-2). Cells were then pelleted and resuspended in 10 ml of SMPP medium (0.3% bovine serum albumin, 5% 2 M sucrose, 25% 4×PAB, 50% 2×SMM), composition of 2×SMM being 1 M sucrose, 0.04 M maleic acid disodium salt hydrate and 0.04 M MgCl₂ (pH 6.5). After the addition of lysozyme (10 mg ml⁻¹) and mutanolysin (75 U ml⁻¹) the mixture was incubated at 37° C. with shaking to generate protoplasts. Protoplast formation was checked microscopically. Protoplasts were then carefully harvested by centrifugation at 5200×g and 4° C. for 5 min, washed twice with ice cold washing electrotransformation buffer (1×SMM) and finally suspended in this solution. Plasmid DNA (1-3 μg) was added to 120 μl of protoplast suspension and the mixture was kept on ice for at least 5 min. The transformation mixture was transferred to a 0.2-cm cuvette and a single electroporation pulse was applied at 25 μF, 400) and 0.7 kV. Immediately after the electroporation shock 1 ml of recovering medium (equal volumes of 4×PAB and 2×SMM, prepared freshly before use) was added to the cuvette. The transformation reaction was then transferred to a 2 ml tube and incubated at 37° C. with shaking for 12 h. For regeneration, the cell suspension was spread on DM3 agar plates (Chang, S. and Cohen, S. (1979) MGG 168(1):111-115) and incubated at 37° C. for 48 h. DM3 regeneration medium contained the following sterile solutions per liter: 200 ml 4% agar, 100 ml 5% casamino acids, 50 ml 10% yeast extract, 100 ml 3.5% K₂HPO₄ and 1.5% KH₂PO₄, 25 ml 20% glucose, 20 ml 1 M MgCl₂, 500 ml 0.5 M sorbitol and 5 ml filter sterilized 2% bovine serum albumin (added to the mixture when the temperature is below 55° C.) and was supplemented with the appropriate antibiotic.

Electroporation of B. subtilis was done according to a modified protocol from Zhang et al. (2011) provided by MoBiTec GmbH (Zhang, G., Bao, P., Zhang, Y., Deng, A., Chen, N. and Wen, T. (2011) Anal. Biochem., 409:130-137). A 2×YT overnight culture was diluted 100-fold with fresh 2×YT medium and the culture was grown to an OD₆₀₀ of 0.2 at 37° C. on a rotary shaker. Then the culture was supplemented with 1% DL-threonine, 2% glycine, 0.1% tryptophan and 0.03% Tween 80. After cultivation for another 60 min, the cell suspension was cooled on ice for 20 min, centrifuged at 5000×g for 10 min at 4° C. and washed twice with electroporation buffer (0.5 M trehalose, 0.5 M sorbitol, 0.5 M mannitol, 0.5 mM MgCl₂, 0.5 mM K₂HPO₄, 0.5 mM KH₂PO₄, pH 7.4, filter-sterilized and stored frozen). Finally, cells were resuspended in electroporation buffer at 1/100 of the original culture volume and 100 μl of cell suspension was mixed with DNA. The transformation mixture was transferred to a 0.1-cm cuvette and electroporation was performed at 1.8 kV with a single pulse delivered with a MicroPulser™ device (Bio-Rad). Immediately after pulse delivery, 1 ml 2×YT broth containing 0.5 M sorbitol and 0.38 M mannitol was added to the cuvette. The transformation suspension was transferred to a 2 ml tube and incubated at 37° C. for 3 h on a rotary shaker. The cells were spread on a selective 2×YT agar plate and incubated at 37° C. overnight.

Using an alternative electroporation protocol (Xue, G. P., J. S. Johnson, and B. P. Dalrymple: 1999; Journal of Microbiological Methods 34:183-191), 5 ml of LB medium containing 0.5 M glucitol was inoculated with B. subtilis and incubated overnight at 37° C. Subsequently, the overnight culture was diluted (1:16) by 75 ml of LB containing 0.5 M glucitol and incubated until an OD₆₀₀ of 0.85-0.95 was obtained. The cells were then pelleted by centrifugation for 10 min at 4° C. at 5.000×g and washed four times by ice-cooled electroporation buffer (10% glycerol, 0.5 M glucitol, 0.5 M mannitol). Finally, the cells were resuspended in 1-2 ml electroporation buffer. Electroporation was performed using 60 μl of competent cells with DNA in a cooled electroporation cuvette (1-mm electrode gap). The cell-DNA mixture was subjected to a single electrical pulse at 25 μF, 200Ω and 21 kV/cm. Finally, 1 ml recovery broth (LB containing 0.5 M glucitol and 0.38 mannitol) was added to the electropermeabilized cells and the bacterial culture was incubated for 3 h at 37° C. followed by plating on LB agar supplemented by an antibiotic.

Two different rich media were used, namely Luria Broth (LB) and 2×YT.

Luria Broth (LB) medium consisted of 1% tryptone, 0.5% yeast extract and 0.5% NaCl (pH 7.2).

2×YT medium consisted of 1.6% tryptone, 1% yeast extract and 0.5% NaCl (pH 7.5).

To prepare rich-medium agar plates 15 g L⁻¹ agar were added.

For shaking flask experiments, Spizizen's minimal medium (Spizizen, J. 1958 Proc. Natl. Acad. Sci. U.S.A 44(10):1072-1078) was used.

Spizizen's minimal medium contains the following salts: 2 g/L (NH₄)₂SO₄, 14 g/L K₂HPO₄, 6 g/L KH₂PO₄, 1 g/L Na₃ citrate×2.H₂O and 0.2 g/L MgSO₄×7.H₂O.

The preculture medium consisted of Spizizen's minimal salts supplemented with 2% D-glucose, 0.05% casamino acids and MgSO₄ to a final concentration of 2 mM (optionally additionally supplemented with biotin and/or L-tryptophan).

The main culture medium consisted of Spizizen's minimal salts supplemented with 2% D-glucose, 0.05% casamino acids, MgSO₄ to a final concentration of 2 mM and 0.5 mL·L⁻¹ 1000× trace element solution (optionally additionally supplemented with biotin and/or L-tryptophan).

Trace element solution (1000×) consisted of 100.6 g/L C₆H₉NO₆, 56.4 g/L ammonium ferric citrate, 9.8 g/L MnCl₂×4.H₂O, 1.6 g/L COCl₂×6.H₂O, 1 g/L CuCl₂×2.H₂O, 1.9 g/L H₃BO₃, 9 g/L ZnSO4×7.H₂O, 1.1 g/L Na₂MoO₄×2.H₂O, 1.5 g/L Na₂SeO₃, 1.5 g/L NiSO₄×6.H₂O.

If necessary, the appropriate antibiotic(s) were added to the medium to make it selective.

B. subtilis strains were initially grown on rich-media agar plates to obtain single colonies. These plates were grown for 1 day at 30-37° C. For shaking flask experiments, a 20-ml preculture was inoculated with a single colony and grown over night at 30-37° C. on a rotary shaker. Subsequent 20-ml main cultures were inoculated with this preculture to a starting OD₆₀₀ of about 0.1 and incubated at 30-37° C. on a rotary shaker. If induction was required, a 40-60 ml main culture was splitted into 20-ml proportions at the timepoint of induction. The culture volume did not exceed 20% of the shaking flask capacity.

### Example 2: Construction of a Bacillus subtilis Production Strain for 3′-Sialyllactose

For the synthesis of 3′-sialyllactose from the metabolic intermediate UDP-N-acetylglucosamine using the neuCBA pathway, a B. subtilis expression plasmid (SEQ ID NO: 1) was constructed. Firstly, the genes neuA, neuB, neuC as well as a α-2,3-sialyltransferase were codon-optimized for expression in B. subtilis and prepared synthetically by GenScript Corp. The Campylobacter jejuni gene neuA (UniProtKB accession number: Q93MP7) encodes a CMP-Neu5Ac synthetase. The Campylobacter jejuni gene neuB (UniProtKB accession number: Q93MP9) encodes a sialic acid synthase. The Campylobacter jejuni gene neuC (UniProtKB accession number: Q93MP8) encodes a N-acetylglucosamine-6-phosphate 2-epimerase. The Pasteurella multocida gene siaT (UniProtKB accession number: Q9CLP3) encodes an α-2,3-sialyltransferase. The open reading frame of the E. coli gene lacY (Gen Bank accession number: NP_414877.1), encoding a lactose permease, was amplified by PCR from chromosomal DNA. Afterwards, the expression cassette <P_{grac100}-neuBCA-siaT-lacY-terminator> was constructed, that comprises all necessary genes under the control of the inducible promoter P_{grac100}. For this, the B. subtilis expression vector pHT253 (MoBiTec GmbH, Göttingen, Germany) was used as backbone. Each gene within the expression cassette was linked to a B. subtilis RBS sequence. Additionally, a suitable B. subtilis terminator sequence from the iGem part repository (sequence ID: BBa_B0015) was placed downstream of the expression cassette. The resulting plasmid <pHT253-P_{grac100}-neuBCA-2,3siaT-lacY-terminator> (SEQ ID NO: 1) was used to transform a sporulating B. subtilis strain (table 1) by exploiting its natural competence.

Gene expression was confirmed by targeted proteomics and/or by real-time PCR.

Transformants were cultivated under conditions that are permissive for B. subtilis to produce 3′-sialyllactose in the presence of exogenous lactose.

### Example 3: Construction of a Bacillus subtilis Production Strain for 3′-Sialyllactose

For the synthesis of 3′-sialyllactose from the metabolic intermediate UDP-N-acetylglucosamine using the neuCBA pathway, a constitutive B. subtilis expression plasmid (SEQ ID NO: 2) was constructed. Firstly, the genes neuA, neuB, neuC and siaT were codon-optimized for expression in B. subtilis and prepared synthetically by GenScript Corp. The Campylobacter jejuni gene neuA (UniProtKB accession number: Q93MP7) encodes a CMP-Neu5Ac synthetase. The Campylobacter jejuni gene neuB (UniProtKB accession number: Q93MP9) encodes a sialic acid synthase.

The Campylobacter jejuni gene neuC (UniProtKB accession number: Q93MP8) encodes a N-acetylglucosamine-6-phosphate 2-epimerase. The Pasteurella multocida gene siaT (UniProtKB accession number: Q9CLP3) encodes an α-2,3-sialyltransferase. The open reading frame of the E. coli gene lacY (Gen Bank accession number: NP_414877.1), encoding a lactose permease, was amplified by PCR from chromosomal DNA. Afterwards, the expression cassette <P43-neuBCA-P_{lepA}-siaT-IacY-terminator> was constructed, that comprises all necessary genes operably linked to the two strong constitutive B. subtilis promoters P_{lepA} and/or P43. The B. subtilis expression vector pHT253 (MoBiTec GmbH, Göttingen, Germany) was used as plasmid backbone. Each gene within the expression cassette was linked to a B. subtilis RBS sequence. Additionally, a suitable B. subtilis terminator sequence from the iGem part repository (sequence ID: BBa_B0015) was placed downstream of the expression cassette.

The resulting plasmid <pHT253-P43-neuBCA-P_{lepA}-siaT-lacY-terminator> (SEQ ID NO: 2) was used to transform spore-forming and non-sporulating B. subtilis strains (Table 1) by exploiting their natural competence.

Gene expression was confirmed by targeted proteomics and/or by real-time PCR.

Transformants were cultivated under conditions that are permissive for B. subtilis to produce 3′-sialyllactose in the presence of exogenous lactose.

### Example 4: Construction of a Bacillus subtilis Production Strain for 6′-Sialyllactose

For production of 6′-sialyllactose in B. subtilis the α-2,6-sialyltransferase from Photobacterium leiognathi was used. The open reading frame of the gene siaT (UniProtKB accession number: DOVYB7) was codon-optimized for expression in B. subtilis and prepared synthetically by GenScript Corp.

The expression plasmid <pHT253-P_{grac100}-neuBCA-2,6siaT-lacY-terminator> (SEQ ID NO: 3) was constructed as described in example 2. The resulting plasmid (SEQ ID NO: 3) was used to transform spore-forming and non-sporulating B. subtilis strains (Table 1) by exploiting their natural competence. Gene expression was confirmed by targeted proteomics and/or by real-time PCR. Transformants were cultivated under conditions that are permissive for B. subtilis to produce 6′-sialyllactose in the presence of exogenous lactose.

### Example 5: Construction of a Bacillus subtilis Strain for Production of 3′-Sialyllactose

Metabolic engineering of B. subtilis (Table 1) was achieved by integration of the heterologous genes Campylobacter jejuni neuA, E. coli nanT and Haemophilus parahaemolyticus siaT and simultaneous deletion of the endogenous gene ganA by homologous recombination. The B. subtilis gene ganA (yvfN, lacA), which is positioned within the galactan operon, encodes a beta-galactosidase.

For the production of 3′-sialyllactose from exogenous sialic acid/N-acetylneuraminic acid and lactose, the open reading frames of neuA, nanT and siaT were operably linked to the B. subtilis constitutive promoter P43 (iGem part repository: sequence ID: BBa_K143013) as an operon. For this purpose, firstly, the genes neuA, nanT and siaT were codon-optimized for expression in B. subtilis and prepared synthetically by GenScript Corp. The Campylobacter jejuni gene neuA (UniProtKB accession number: Q93MP7) encodes a CMP-Neu5Ac synthetase, the E. coli gene nanT (GenBank accession number: NP_417691.4) encodes a sialic acid transporter and the H. parahaemolyticus gene siaT (UniProtKB accession number: I3DHL4) encodes an alpha-N-acetylneuraminyl-2,3-beta-galactosyl-1,3-N-acetylgalactosaminidase 6-alpha-sialyltransferase (alpha-2,3-sialyltransferase).

Each gene within the expression cassette was linked to a B. subtilis RBS sequence. Additionally, a suitable B. subtilis terminator sequence from the iGem part repository (sequence ID: BBa_B0015) was placed downstream of the expression cassette.

The cloning vector pBR322 (New England Biolabs GmbH, Frankfurt, Germany) was used as plasmid backbone. The complete integration cassette was assembled to create the suicide plasmid <pBR322 flank ganA up-P43-siaT-neuA-nanT-terminator-erm-flank ganA down> (SEQ ID NO: 4). Afterwards, B. subtilis was transformed with this plasmid by its natural competence. Cells were spread on 2×YT agar plates containing the appropriate antibiotic (5 μg mL⁻¹ erythromycin). Integration of the expression cassette <P43-siaT-neuA-nanT-terminator> into the ganA locus of the B. subtilis genome, yielding strain A, was verified by colony PCR. Gene expression was confirmed by targeted proteomics and/or by real-time PCR.

For import of exogenous lactose into the Bacillus cell, the E. coli gene lacY was integrated into the endogenous amyE (amyA) locus of the B. subtilis genome (encoding for an alpha-amylase). For this purpose, the open reading frame of the E. coli lacY gene (Gen Bank accession number: NP_414877.1), encoding a lactose permease, was amplified by PCR from chromosomal DNA. The integration cassette <flank amyE up-lox71-aad9-lox66-P43-lacY-flank amyE down> (SEQ ID NO: 5) was constructed and cloned into pBR322 (New England Biolabs GmbH, Frankfurt, Germany). Strain A was transformed with the resulting suicide plasmid by natural competence. Integration of the expression cassette <P43-lacY> into the amyE locus of strain A, yielding the final strain B, was verified by colony PCR.

Gene expression was confirmed by targeted proteomics and/or by real-time PCR. Strain B was cultivated under conditions that are permissive for B. subtilis to produce 3′-sialyllactose in the presence of exogenous lactose and sialic acid/N-acetylneuraminic acid.

### Example 6: Production of Sialyllactose Using Metabolically Engineered Bacillus subtilis Strains

A preculture was inoculated with a metabolically engineered Bacillus subtilis strain suitable for the biosynthesis of sialyllactose (as described in examples 2-5).

The preculture was incubated at 30-37° C. over night and then diluted to a starting OD₆₀₀ of about 0.1 in fresh main culture medium. When the main culture reached an OD₆₀₀ of approximately 0.5, 2 mM lactose was added to the growth medium. When the inducible promoter P_{grac100} was used for gene expression, induction was carried out with lactose (2 mM) or with both lactose (2 mM) and IPTG (1 mM). For the salvage biosynthesis of CMP-N-acetylneuraminic acid, additionally, 2 mM sialic acid/N-acetylneuraminic acid were added. Cultivation was discontinued after about 24 h/48 h after induction and intracellular and extracellular sialyllactose was analyzed by thin layer chromatography and/or HPLC and/or mass spectrometry (as described in WO 2017/042382 A or WO 2019/008133 A). Biosynthesis of substantial amounts of sialyllactose (3′-sialyllactose/6′-sialyllactose) was detectable.

1. A non-sporulating Bacillus cell for the production of a sialylated oligosaccharide, wherein said Bacillus cell has been genetically engineered to possess a lactose permease, a CMP-NeuNAc biosynthesis pathway and a sialyltransferase.

2. The Bacillus cell according to claim 1, wherein the sporulation capability of the Bacillus cell has been impaired by deletion or functional inactivation of one or more of the genes encoding Spo0A, sigma E and sigma F.

3. The Bacillus cell according to claim 1 or 2, wherein the lactose permease is E. coli LacY or a functional variant thereof.

4. The Bacillus cell according to any one of claims 1 to 3, wherein the CMP-NeuNAc biosynthesis pathway uses GlcN-1P or GlcNAc-6P as intermediate.

5. The Bacillus cell according to any one of claims 1 to 4, wherein the CMP-NeuNAc biosynthesis pathway uses a sialic acid salvage pathway.

6. The Bacillus cell according to any one of claims 1 to 5, wherein the sialyltransferase is a lactose-accepting sialyltransferase, preferably a sialyltransferase selected from the group consisting of α-2,3-sialyltransferases, α-2,6-sialyltransferases and α-2,8-sialyltransferases.

7. The Bacillus cell according to any one of claims 1 to 6, wherein said Bacillus cell lacks any β-galactosidase activity or possesses reduced β-galactosidase activity as compared to a wild-type progenitor Bacillus cell of the same species.

8. The Bacillus cell according to claim 7, wherein the Bacillus cell has been genetically engineered by deletion or functional inactivation of at least one of the genes selected from the group consisting of yesZ and ganA.

9. The Bacillus cell according to any one of claims 1 to 8, wherein said Bacillus cell is a Bacillus subtilis cell.

10. The Bacillus cell according to any one of claims 1 to 9, for use in the production of 3′-sialyllactose, wherein the CMP-NeuNAc biosynthesis pathway uses GlcN-1P as intermediate, and wherein the sialyltransferase is a α-2,3-sialyltransferase.

11. The Bacillus cell according to any one of claims 1 to 9, for use in the production of 6′-sialyllactose, wherein the CMP-NeuNAc biosynthesis pathway uses GlcN-1P as intermediate, and wherein the sialyltransferase is a α-2,6-sialyltransferase.

12. Use of a Bacillus cell according to any one of claims 1 to 11 for the production of a sialylated oligosaccharide.

13. A method for the production of a sialylated oligosaccharide, the method comprising
providing a non-sporulating Bacillus cell as defined in any one of claims 1 to 11;
cultivating said Bacillus cell in a fermentation broth and under conditions that are permissive for the production of a sialylated oligosaccharide; and optionally
retrieving the sialylated oligosaccharide from the medium and/or the Bacillus cell.

14. The method according to claim 13, wherein the fermentation broth contains lactose.

15. A sialylated oligosaccharide produced by a method according to claim 13 or 14.

16. Use of a sialylated oligosaccharide according to claim 15 for the manufacture of a nutritional composition, preferably of an infant formula.

17. A nutritional composition containing a sialylated oligosaccharide according to claim 15.
